(19) United States
(12) Patent Application Publication
(10) Pub. No.: US 2005/0207981 A1
(43) Pub. Date: Sep. 22, 2005

(54) Compositions and methods for determining substrate specificity of hydrolytic enzymes

(75) Inventors: Amy M. Barrios, Pasadena, CA (US); Charles S. Craik, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/989,590

(22) Filed: Nov. 15, 2004

Related U.S. Application Data
(60) Provisional application No. 60/519,938, filed on Nov. 14, 2003.

(51) Int. Cl. C12Q 1/68; G01N 33/53; A61K 49/00; C07F 5/00

(52) U.S. Cl. 424/9.361; 435/7.1; 534/15; 424/9.6

(57) ABSTRACT

The present invention relates to a novel compound comprising a detectable moiety covalently linked to a structural moiety. Upon cleavage of the covalent bond linking the two moieties, the detectable moiety becomes capable of complexing a lanthanide ion, and the lanthanide-detectable moiety complex provides a detectable signal. The structural moiety of the compound is a homo- or hetero-multimer of amino acids, nucleotides, or saccharides. A library comprising at least two member compounds with different structural moieties is also provided in this application. Further described are methods for identifying the substrate specificity of a hydrolytic enzyme by using the library of the present invention to determine the preferred structural moiety for any particular enzyme having the potential capability of cleaving the covalent bond between the detectable moiety and the structural moiety of the member compounds, as well as methods for using the novel compound of this invention for detecting in a sample the presence of a pre-determined hydrolytic enzyme, whose preferred substrate specificity is known and represented by the structural moiety of the compound.

### CROSS-REFERENCES TO RELATED APPLICATIONS

This application claims priority to provisional application U.S. Ser. No. 60/519,938, filed Nov. 14, 2003, the contents of which are incorporated by reference in the entirety.

### STATEMENT AS TO RIGHTS TO INVENTIONS MADE UNDER FEDERALLY SPONSORED RESEARCH OR DEVELOPMENT

This work was supported in part by National Institute of Health Grants A135707 and CA72006. The Government may have certain rights in this invention.

### BACKGROUND OF THE INVENTION

The ability of an enzyme to discriminate among many potential substrates is an important factor in maintaining the fidelity of most biological functions. While substrate selection can be regulated on many levels in a biological context, such as spatial and temporal localization of enzyme and substrate, concentrations of enzyme and substrate, and requirement of cofactors, the substrate specificity at the enzyme active site is the overriding principle that determines the turnover of a substrate. Characterization of the substrate specificity of an enzyme clearly provides invaluable information for the dissection of complex biological pathways. Definition of substrate specificity also provides the basis for the design of selective substrates and inhibitors to study enzyme activity.

Of the genomes that have been completely sequenced, 2% of the gene products encode proteases (Barrett, A. J., et al., (1998) Handbook of Proteolytic Enzymes (Academic Press, London)). This family of enzymes is crucial to every aspect of life and death of an organism. With the identification of new proteases, there is a need for the development of rapid and general methods to determine protease substrate specificity. While several biological methods, such as peptides displayed on filamentous phage (Matthews, D. J., et al. (1993) Science 260:1113-7; Ding, L., et al., (1995) Proceedings of the National Academy of Sciences of the United States of America 92:7627-31), and chemical methods, such as support-bound combinatorial libraries (Lam, K. S., et al., (1998) Methods in Molecular Biology, 87:1-6), have been developed to identify proteolytic substrate specificity, few offer the ability to rapidly and continuously monitor proteolytic activity against complex mixtures of substrates in solution.

Knowledge of the primary sequence specificity of a protease provides a first approximation in determining its function in vivo and a number of researchers have developed substrate libraries for this purpose. Of these, the most widely applicable has been the use of coumarin-based fluorogenic substrate libraries to scan the substrate binding pockets on the N-terminal side of the scissile bond (the non-prime⁴ subsites).⁵⁻⁸ Synthetic combinatorial peptide libraries that systematically scan through the P1, P2, P3 and P4 subsites have been developed and used to profile the specificity of serine, cysteine and aspartyl proteases.^{5,6,9-11} In addition to providing insight into the biochemistry of a protease, knowledge of substrate specificity can aid in the design of selective protease inhibitors. Simple chemical substrate libraries can yield a wealth of biochemically and therapeutically relevant data.

In order to develop a fluorogenic peptide library that can be used to probe the prime site binding pockets of a protease, an appropriate fluorophore must be chosen. In the case of the non-prime side coumarin-based fluorogenic libraries, the fluorescence of the coumarin moiety is quenched by attachment through an amide bond to the amino acid chain. Upon cleavage of the coumarin-peptide amide bond, the amino group of the coumarin is released and fluorescence is greatly enhanced, as shown in Equation 1 in FIG. 6. A complementary approach is needed in the development of a fluorophore for use in prime site libraries, wherein cleavage of the peptide-fluorophore bond releases a fluorophore with a free carboxylic acid moiety.

Although the existing libraries have provided valuable information about the specificity of numerous proteases, one of their main shortcomings is the fact that they only profile the non-prime subsites.³ Phage display of peptide substrates,12 FRET-based polypeptide substrates^{13,14} and acyl transfer from protease to substrate¹⁵ have been used to map the prime site specificity of a limited number of proteolytic enzymes. Unfortunately, each of these methods has significant drawbacks such as poor solubility, restricted diversity and limited applicability. A simple chemical library that can be used to profile the prime site specificity of a protease is desirable. Ideally, a system could be designed in which (1) an easily detectable, highly sensitive signal (e.g., fluorescence) is activated upon substrate hydrolysis, (2) cleavage of many substrates can be observed simultaneously and (3) the results can be rapidly deconvoluted to produce a specificity profile. The present invention answers these and other needs.

### SUMMARY OF THE INVENTION

The present invention provides a novel compound for determining enzyme substrate specificity. The compound comprises a detectable moiety and a structural moiety that are covalently linked together. The detectable moiety is a chemical group that does not impart any significant detectable signal until the covalent linkage between the detectable moiety and structural moiety is cleaved by the enzyme. The cleaved detectable moiety then becomes available to interact with a metal ion, such as a lanthanide ion, and form a complex that provides a detectable signal, which can be measured, e.g., in the form of fluorescence or magnetic resonance contrast, and indicates enzymatic activity.

The structural moiety, on the other hand, provides the structural basis of substrate specificity of the enzyme being tested. The structural moiety is typically a oligomer of amino acid, nucleotide, or saccharide residues connected together by covalent linkages such as peptide bond, phosphodiester bond, and the like. The oligomer may be homogenous, i.e., consisting of only one type of residues (such as a strand of polynucleotide, which may or may be limited to same individual nucleotide), or heterogenous, i.e., consisting of two or more different types of residues (such as a combination of amino acids and sugars). After the exposure of a compound of the present invention to a test enzyme under suitable conditions, if a detectable signal from the detectable moiety-metal ion complex is registered, then the structural moiety of the compound is deemed to represent a preferred substrate structural profile for that enzyme. In one preferred embodiment, the compound comprises a peptide as the structural moiety and is useful for determining a protease's substrate specificity C-terminal to the scissile bond. In another preferred embodiment, the detectable moiety is 5-fluorosalicylic acid (fsa), phenanthroline carboxylic acid (pca), or their derivatives. In another preferred embodiment, the metal ion is a lanthanide ion, such as erbium, europium, samarium, dysprosium, or gadolinium.

This invention also provides a library of member compounds that each has the general features of the compound described above and a method for using the library to assess the structural preference for substrate of an enzyme. The library of the invention comprises at least two, but typically more, members having different structural moieties, such that a hydrolytic enzyme (e.g., a protease or an esterase) with previously unknown substrate specificity may be tested for activity among the library members. The substrate specificity of the enzyme is determined based on the structural moiety of the member or members to which a high level of hydrolytic activity by the enzyme is observed. This screening process may be conducted with the individual library members sequentially or it may be performed simultaneously using an addressable array where the identity of a member compound corresponds to its physical location in the array. In one preferred embodiment, the compound comprises a peptide as the structural moiety and is useful for determining a protease's substrate specificity C-terminal to the scissile bond. In another preferred embodiment, the detectable moiety is 5-fluorosalicylic acid (fsa), phenanthroline carboxylic acid (pca), or their derivatives. In another preferred embodiment, the metal ion is a lanthanide ion, such as erbium, europium, samarium, dysprosium, or gadolinium.

Further provided is a method for using the compound of the present invention to detect the presence of a pre-selected enzyme, which has a previously determined structural preference for substrate. This method comprises the following steps: first, contacting, at the presence of a suitable metal ion, a sample in which the presence of this enzyme is being tested with a compound of the present invention. This compound has a structural moiety that fits the substrate specificity of the enzyme; and second, detecting changes in a detectable signal, such as fluorescence or magnetic resonance contrast. An increase in the detectable signal indicates the presence of this enzyme. In one preferred embodiment, the compound comprises a peptide as the structural moiety that fits the profile of a pre-selected protease's substrate specificity C-terminal to the scissile bond. In another preferred embodiment, the detectable moiety is 5-fluorosalicylic acid (fsa), phenanthroline carboxylic acid (pca), or their derivatives. In another preferred embodiment, the metal ion is a lanthanide ion, such as erbium, europium, samarium, dysprosium, or gadolinium.

Other objects and advantages of the present invention will be apparent from the Detailed Description, which follows.

### BRIEF DESCRIPTION OF THE DRAWINGS

FIG. 1 is a diagram of three libraries scanning the P1′, P2′ and P3′ positions in a 96-well format.

FIG. 2 is a graphical display of data from a library scan.

FIG. 3 is a representation of the prime side subsites in bovine α-chymotrypsin. The P1′ and P3′ subsites overlap, resulting in an interdependence between these two sites with Asp35 and Asp64 providing the major specificity determinants. Phe41 lies beneath the plane of the peptide, providing a potential cation-π interaction with the Arg residue in P3′ and its backbone oxygen atom providing a hydrogen bond acceptor for the amide N—H moiety of the P2′ residue. The P2′ pocket is less well defined, with potential hydrogen bond donors and acceptors such as Asn150 and Thr151 available.

FIG. 4 is a representation of the reaction between the phenanthorline carboxylic acid (pca) detectable moiety covalently bound to the tetrapeptide LAAA and bovine alpha chymotrypsin, a proteolytic enzyme. In this graph, the relative rate of substrate cleavage (v), measured by detecting the fluorescence of the cleaved pca moiety coordinated to europium, is on the y axis, and the concentration of the pca-LAAA peptide moiety ([pca-LAAA]) is on the x axis.

FIG. 5 illustrates reaction scheme 1.

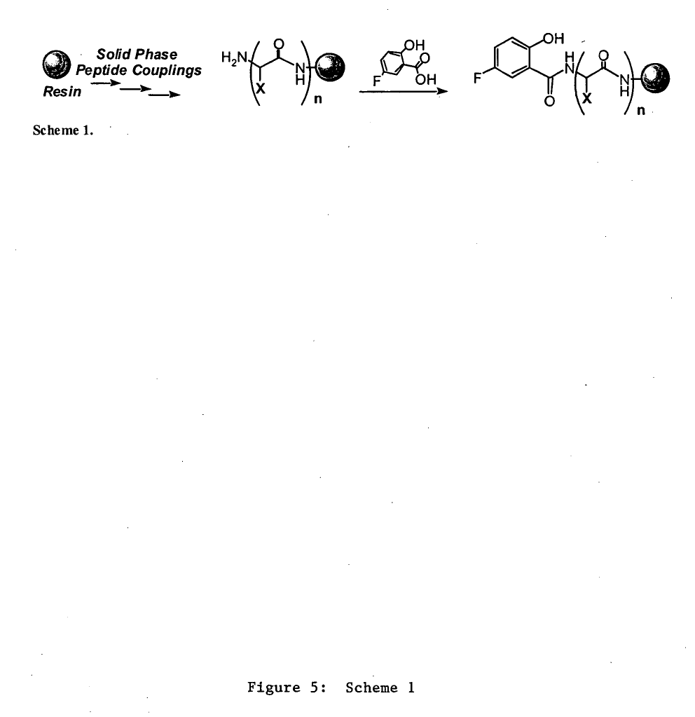

FIG. 6 illustrates Equations 1, 2, and 3, which show exemplary reactions of hydrolysis of a peptide labeled with a detectable moiety and the release of the detectable moiety. The detectable moiety is coumarin in Equation 1, 5-fluorosalicylic acid (fsa) in Equation 2, and phenanthorline carboxylic acid (pca) in Equation 3.

### DETAILED DESCRIPTION OF THE INVENTION AND THE PREFERRED EMBODIMENTS

### ABBREVIATIONS AND DEFINITIONS

All technical and scientific terms used herein generally have the same meaning as commonly understood by one of ordinary skill in the art to which this invention belongs. The present definitions and abbreviations are generally offered to supplement the art-recognized meanings. Generally, the nomenclature used herein and the laboratory procedures organic chemistry, enzyme chemistry and peptide synthesis described below are those well known and commonly employed in the art. Generally, enzymatic reactions and purification steps are performed according to the manufacturer's specifications. Standard techniques, or modifications thereof, are used for chemical syntheses and chemical analyses.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.),VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical” is the group derived from a carboxylic acid by removing the —OH moiety therefrom.

The term “alkyl,” by itself or as part of another substituent means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present invention contain between about one and about twenty five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom selected from the group consisting of O, N, Si, P and S, and wherein the nitrogen, phosphorous and sulfur atoms are optionally oxidized, and the nitrogen heteroatom is optionally be quaternized. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic moiety that can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, tetrazolyl, benzo[b]furanyl, benzo[b]thienyl, 2,3-dihydrobenzo[1,4]dioxin-6-yl, benzo[1,3]dioxol-5-yl and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) is meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, (═O), ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, (═O), ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

Two of the substituents on adjacent atoms of the aryl or heteroaryl ring may optionally be replaced with a substituent of the formula —T—C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O— —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “amino” or “amine group” refers to the group —NR′R″ (or —N⁺RR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —N⁺RR′R″ and its biologically compatible anionic counterions. The term “halogen” is used herein to refer to fluorine, bromine, chlorine and iodine atoms.

The term “hydroxy” is used herein to refer to the group —H.

The term “amino” is used to —NRR′, wherein R and R′ are independently H, alkyl, aryl or substituted analogues thereof. “Amino” encompasses “alkylamino” denoting secondary and tertiary amines and “acylamino” describing the group RC(O)NR′.

The term “alkoxy” is used herein to refer to the —OR group, where R is alkyl, or a substituted analogue thereof. Suitable alkoxy radicals include, for example, methoxy, ethoxy, t-butoxy, etc.

“Carrier molecule,” as used herein refers to any molecule to which a compound of the invention is attached. Representative carrier molecules include a protein (e.g., enzyme, antibody), glycoprotein, peptide, saccharide (e.g., mono- oliogo- and poly-saccharides), hormone, receptor, antigen, substrate, metabolite, transition state analog, cofactor, inhibitor, drug, dye, nutrient, growth factor, etc., without limitation. “Carrier molecule” also refers to species that might not be considered to fall within the classical definition of “a molecule,” e.g., solid support (e.g., synthesis support, chromatographic support, membrane), virus and microorganism.

As used herein, the term “linking group” refers to a group that links a detectable moiety to a solid support. Linking groups of diverse structures are useful in practicing the present invention. Exemplary linking groups include, but are not limited to, organic functional groups (e.g., —C(O)—, —NR—, —C(O)S—, —C(O)NR—, etc.); substituted or unsubstituted alkyl, substituted or unsubstituted heteroalkyl and substituted or unsubstituted aryl groups each of which are, in addition to other optional substituents, homo- or hetero-disubstituted with organic functional groups, that adjoin the linker arm to the fluorophore and to the solid support. The linking groups of the invention can include a group that is cleaved by, for example, light, heat, reduction, oxidation, hydrolysis or enzymatic action (e.g., nitrophenyl, disulfide, ester, etc.). Alternatively, the linking group is substantially stable under a range of conditions. By providing for the use of linkers with a wide range of physicochemical characteristic, the invention allows selected properties of the material of the invention and its conjugates to be manipulated. Properties that are amenable to manipulation include, for example, hydrophobicity, hydrophilicity, surface-activity and the distance from the solid support of the species bound to the solid support via the linking group.

“Peptide” refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

“Fluorogen,” as used herein, refers broadly to a class of compounds capable of being modified enzymatically or otherwise to give a derivative fluorophore, which has a modified or an increased fluorescence. In a specific example, a fluorogen is a species with metal chelating properties. When combined with an appropriate metal ion, the fluorogen chelates the metal ion, producing a fluorescent metal complex.

“Solid support,” as used herein refers to a material that is substantially insoluble in a selected solvent system, or which can be readily separated (e.g., by precipitation) from a selected solvent system in which it is soluble. Solid supports useful in practicing the present invention can include groups that are activated or capable of activation to allow selected species to be bound to the solid support. A solid support can also be a substrate, for example, a chip, wafer or well, onto which an individual, or more than one compound, of the invention is bound.

“Organic functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Methods to prepare each of these functional groups are well-known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

A “detectable moiety” and a “structural moiety” are two essential portions of a claimed compounds of the present invention. When covalent linked to a structural moiety, such as by a peptide bond or an ester bond, a detectable moiety does not impart any significant detectable signal; once the covalent bond joining the two moieties is cleaved, such as by enzymatic action, the detectable moiety leaves the structural moiety and becomes available to engage a metal ion, such as a lanthanide ion, and form a chelating complex with the metal ion. Subsequently, the resulting complex imparts a detectable signal, which may be measured by, e.g., an increase in fluorescence emission or an increase in magnetic resonance contrast. Some exemplary detectable moieties include 5-fluorosalicylic acid (fsa), phenanthroline carboxylic acid (pca), and their derivatives.

A “structural moiety” in this context refers to a portion of the claimed compound that provides a structural basis for the substrate preference of a hydrolytic enzyme. The structural moiety is an oligomer preferably consisting of one, two, three, or more residues of amino acid, nucleotide, or saccharide. In some cases, an oligomer consists of two to four residues; in other cases, an oligomer consists of two to six residues. An oligomer often has no more than ten residues. The chemical structure of an oligomer within the meaning of the present application can vary, depending on the enzyme of interest. For instance, the structural moiety in a compound used for determining substrate specificity of a protease is preferably a peptide consisting of one, two, or more amino acid residues.

A “detectable signal” in this application refers to a signal detectable by spectroscopic, photochemical, biochemical, immunochemical, electrical, magnetic, optical, or chemical means. Exemplary signals include fluorescent, radioactive, enzymatic, magnetic, and calorimetric signals. Preferably, the detectable signals useful for this invention are fluorescent and magnetic resonance signals.

As used herein, “magnetic resonance contrast” refers to a change in the nuclear magnetic resonance signal of the sample upon enzymatic cleavage of the substrate. In a specific example, enzymatic cleavage of the substrate releases a chelating group, which then coordinates to a lanthanide ion such as gadolinium(III) in the reaction mixture, changing the relative relaxation rates of the hydrogen nuclei in the vicinity of the metal complex.

The term “amino acid” refers to naturally-occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

A “nucleic acid” as used herein encompasses all natural or artificial nucleotides or deoxynucleotides, their analogues, mimetics, and derivatives.

A “saccharide” as used herein encompasses all forms of simple or complex sugars conventionally regarded as saccharide in the art. For example, a “saccharide” in this application may be a monosaccharide or a disaccharide. This term also encompasses sugars of any natural or artificial configurations.

An “oligomer” as used herein refers to a polymeric chemical structure that consists of one or more monomer units connected to each other by covalent bonds. The preferred monomer units include amino acids, nucleotides, and saccharides. An oligomer in this application may be a homo-oligomer, i.e., consists of only one type of monomer units (e.g., every monomer is an amino acid, which may or may not be the same individual amino acid throughout the entire oligomer), or a hetero-oligomer, i.e., consists of a mixture of at least two different types of monomer units (e.g., amino acid residues combined with sugar residues).

The term “increase,” as used herein, refers to any detectable positive change in quantity of a parameter when compared to a standard or a control. The level of this change, for example, in the intensity of fluorosence or magnetic resonance contrast, is preferably at least 10% or 20%, and more preferably at least 30%, 40%, 50%, 60% or 80%, and most preferably at least 100% above the control.

An “addressable array” as used herein refers to a solid support that provides more than one site or location having a compound of the present invention bound thereto, where there exists a defined correlation between any one given site and the distinct identity of the compound located at that site.

Compounds

The present invention provides a compound that comprises a structural moiety conjugated to a detectable moiety. The detectable moiety is converted to a chemical entity capable of chelating a metal ion upon being cleaved from the structural moiety. In one preferred embodiment, the compound comprises a peptide as the structural moiety that is covalently linked to a detectable moiety, fsa, through a peptide bond. Upon cleavage of the peptide bond by a protease that recognizes the peptide sequence of the structural moiety as its preferred prime side sequence, fsa is released and, when contacted with a terbium ion, chelates the terbium ion to form a fsa-terbium complex that emit fluorescence upon proper excitation.

In a first aspect, the compound of the invention has the formula:
R—X-A¹-A²-(Aⁱ)_{J-2} (I).

R is the detectable moiety; A¹-A²-(Aⁱ)_{J-2} is the structural moiety consisting of an oligomer of amino acid, nucleotide, or saccharide residues; R is a substituted or unsubstituted aryl or substituted or unsubstituted heteroaryl moiety; X is a member selected from the group consisting of C(O)—NH, C(O)—O, and OP(O)(OH)—O; each of A¹ through Aⁱ is an amino acid, a nucleotide, or a saccharide residue;

J denotes the number of residues forming the homo-oligomer and is a member selected from the group consisting of the numbers from 2 to 10, such that J−2 is the number of residues in the oligomer sequence exclusive of A¹-A²; and i denotes the position of the residue relevant to A¹ and when J is greater than 2, i is a member selected from the group consisting of the numbers from 3 to 10.

In generally preferred embodiment, R is a detectable moiety that is capable of chelating a metal ion, particularly a lanthanide ion, to form fluorescent complexes. Such exemplary detectable moieties include fsa and pca, which can chelate lanthanide ions including terbium, europium, samarium, dysprosium, or gadolinium.

In a second aspect, the invention provides a compound having the formula:

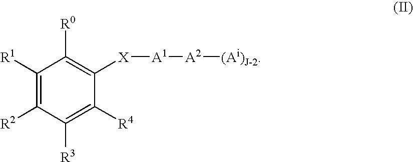

In this formula, R¹, R², R³, and R⁴ are independently selected from the group consisting of H, halogen, —NO₂, —CN, —C(O)ₘR⁵, —C(O)NR⁶R⁷, —S(O)ₜR⁸, —SO₂NR⁹R¹⁰, —OR¹¹, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS.

R⁰, R⁵, R⁶, R⁷, R⁸, R⁹, R¹⁰, and R¹¹ are independently selected from the group consisting of H, substituted or unsubstituted alkyl, and substituted or unsubstituted heteroalkyl;

R¹² is either present or absent, and when present, is a member selected from the group consisting of substituted or unsubstituted alkyl, and substituted or unsubstituted heteroalkyl; when R¹² is absent, Y is attached directly to the detectable moiety.

R¹³ is a linking group adjoining the detectable moiety and the solid support; m is a member selected from the group consisting of the integers from 1 to 2; t is a member selected from the group consisting of the integers from 0 to 2; Y is an organic functional group or methyl, and is a member selected from the group consisting of —COOR¹⁴, CONR¹⁴R¹⁵, —C(O)R¹⁴, —OR¹⁴, —SR¹⁴, NR¹⁴R¹⁵, C(O)¹⁴R¹⁵, and —C(O)SR¹⁴; R¹⁴ and R¹⁵ are members independently selected from the group consisting of H, substituted or unsubstituted alkyl, substituted or unsubstituted heteroalkyl, substituted or unsubstituted aryl and substituted or unsubstituted heteroaryl; and SS is a solid support.

In an exemplary embodiment, the compound of the invention has the formula: 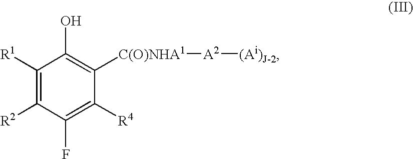
in which the identities of R¹-R⁴ are as described in the context of Formula II.

In another exemplary embodiment, the invention provides a compound having the formula:
R—C(O)—NH-A (IV),
in which A is an amino acid residue selected from the group consisting of natural amino acids, unnatural amino acids, and modified amino acids. The identity of R is as described in the context of Formula I.

In another exemplary embodiment, the compound has the formula: 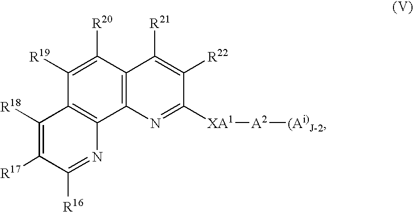
in which R¹⁶ to R²² is independently selected from the group consisting of are independently selected from the group consisting of H, halogen, —NO₂, —CN, —C(O)ₘR⁵, —C(O)NR⁶R⁷, —S(O)ₜR⁸, —SO₂NR⁹R¹⁰, —OR¹¹, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS; and R⁵, R⁶, R⁷, R⁸, R⁹, R¹⁰, and R¹¹ are independently selected from the group consisting of H, substituted or unsubstituted alkyl, and substituted or unsubstituted heteroalkyl.

In another exemplary embodiment, the compound has the formula:

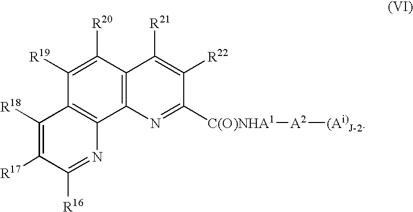

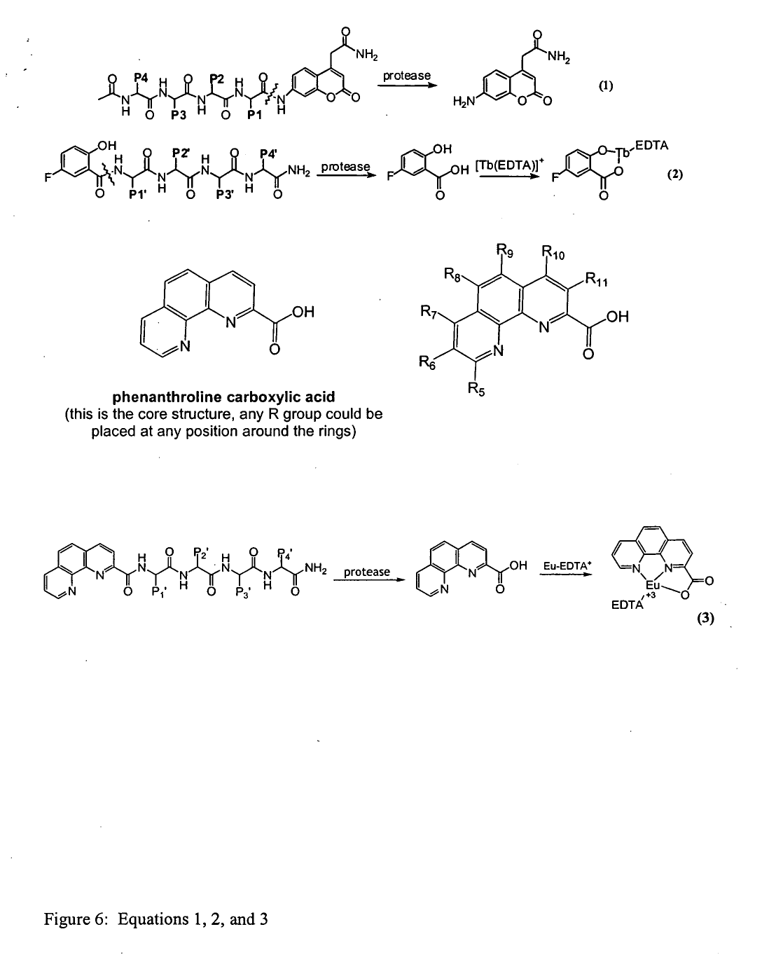

In a further exemplary embodiment, the detectable moiety is based upon the 5-fluorosalicylic acid (fsa) moiety. In this embodiment, the structural moiety is hydrolyzed and the resulting free fsa ligand coordinates to a terbium ion in the reaction solution to produce a fluorescent signal (Equation 2 of FIG. 6). The intrinsic fluorescence of the lanthanide ion terbium(III) is very weak, but can be enhanced by the use of laser excitation or a sensitizing ligand.¹⁶ Because non-radiative decay of the lanthanide ion excited state can occur through vibronic coupling to water molecules bound to the metal ion, an EDTA ligand or another similar shielding ligand is required to exclude water from the metal coordination sphere.¹⁷

In another preferred embodiment, the detectable moiety is based on a phenanthroline carboxylic acid (pca) moiety, which upon hydrolysis from the structural moiety becomes available for chelating europium and produces a fluorescent signal (Equation 3 of FIG. 6).

The compounds of the invention are generally of use as solid supports for the synthesis of individual compounds besides the exemplary detectable moiety-conjugated peptides and libraries consisting of a collection or an array of such individual compounds. Exemplary compounds that can be synthesized using the solid support of the invention include, but are not limited to, small molecules and oligomers (e.g., nucleic acids, lipids, saccharides, etc.). Thus, the present invention provides libraries of a broad class of compounds comprising a detectable moiety and a structural moiety generally described above.

The compounds of the present invention are of use as probes for a variety of applications, including structural elucidation of materials, substrate specificity of enzymes, hybridization of nucleic acids, substrate transformation, digestion or degradation of biomolecules, such as peptides, nucleic acids, saccharides and the like. As discussed above, the present invention provides a solid support, which allows for the conjugation of a detectable moiety to compounds of different types, which are synthesized on the solid support of the invention.

In some examples, the compounds of the present invention have a peptide sequence that includes at least one peptide bond cleavable by an enzyme, preferably a protease. Cleaving the peptide bond preferably releases the detectable moiety from the peptide sequence, thereby producing a free detectable moiety and a peptide moiety. The peptide bond, which undergoes enzymatic cleavage can be located at any site within the peptide sequence, but is preferably located at a peptide bond formed between a carboxylic acid moiety of the detectable moiety and an amine of the peptide amino terminus.

In protease studies, the present invention provides the ability to introduce an additional element of diversity in the positional scanning combinatorial libraries through the preparation of a library of structural moieties (peptide sequences) consisting of a plurality of wells (pre-selected amino acids can be omitted or included) addressing a fixed P1′ amino acid. In an illustrative embodiment having 20 wells, a tetrapeptide is prepared in which the P2′-P3′-P4′positions in the library consist of an equimolar mixture of 19 amino acids (cysteine is omitted and norleucine is substituted for methionine) for a total of 6,859 substrates per well and 130,321 substrates per library. The present invention provides a further advantage in that, if members of the library are sparingly soluble under a particular set of conditions, to avoid insolubility of the substrates as well as to maintain kcat/Km conditions, the concentration for each individual substrate per well can be decreased to approximately 0.01 μM.

The present invention also provides methods useful for assessing the activity of and providing the profile of substrate specificity of enzymes other than proteases. For example, the detectable moiety described above can be linked to a peptide, a mono- or oligosaccharide, a polynucleotide, or other chemical moiety through an ester linkage. Enzymatic cleavage of the ester bond would release the detectable moiety, and an increase in fluorescence or magnetic resonance contrast would be detected according to methods described herein or known to those skilled in the art. Alternatively, the detectable moiety can be linked to any appropriate molecule using a variety of chemical linkages well-known to those familiar with the art using art-recognized methods.

Libraries

The synthesis and screening of chemical libraries to identify compounds having useful biological and material properties is now a common practice. Illustrative of the many different types of libraries that have been prepared are libraries including collections of oligonucleotides, oligopeptides, and small or large molecular weight organic or inorganic molecules. See, Moran et al., PCT Publication WO 97/35198, published Sep. 25, 1997; Baindur et al., PCT Publication WO 96/40732, published Dec. 19, 1996; Gallop et al., J. Med. Chem. 37:1233-51 (1994).

In a further aspect, the present invention provides a library of member compounds having a structure according to Formula I. The library includes at least a first compound having a first structural moiety covalently attached to a first detectable moiety and a second compound having a second structural moiety covalently attached to a second fluorogenic moiety. For each of the members of the library, R is independently selected from detectable moieties according to the description of Formula I. Thus, the detectable moiety can be the same for each of the compounds of a particular library or the structure of the detectable moiety can vary in a selected manner for two or more member compounds of the library.

In an exemplary embodiment, there is provided a peptide library that includes detectable moiety-conjugated peptides according to Formula I, II, III, VI, V, or VI. In another exemplary embodiment, for each peptide of the library, at least one of R⁰, R¹, R², R³, and R⁴ is a member independently selected from —R¹⁴—SS and —R²⁰—Y.

As discussed above, each of the peptide sequences and peptide lengths of the peptides of a particular peptide library are independently selected. Thus, in a preferred embodiment, each of peptides of the library is characterized by a peptide sequence that is different than the peptide sequence of each of the other peptides. The difference resides in peptide sequence, peptide length or both. Thus, a preferred library of the invention is one wherein, an amino acid residue selected from at least one member of A¹, A² . . . Aⁱ of the first peptide is a different amino acid residue than an amino acid residue at a corresponding position relative to A¹ of the second peptide.

The peptide libraries of the invention are broadly characterized by the presence of peptides of diverse structure within the library. In an exemplary embodiment, the diversity in the peptides of the library is provided by peptide sequences that have different amino acid residues at A¹. Those of skill in the art will appreciate that the focus of the present discussion on diversity at A¹ is for clarity of illustration and is not intended to exclude those peptide sequences having diversity at positions other than A¹ or those peptide sequences having diversity at positions in addition to A¹.

Thus, in a preferred embodiment, the exemplary peptide library is characterized by having at least six peptides having different peptide sequences wherein, A¹ is a different amino acid residue in each of the different peptide sequences. In another preferred embodiment, the library includes at least twelve peptides, and more preferably twenty peptides having different peptide sequences, in which A¹ is a different amino acid residue in each of the different peptide sequences.

The amino acid residue at A¹ can be any amino acid residue selected from the group consisting of natural amino acids, unnatural amino acids and modified amino acids. In a preferred embodiment, A¹ is a member selected from the group consisting of Lys, Arg, and Leu.

The peptides of the library can have a peptide sequence of substantially any useful length for a selected purpose. Presently preferred peptide sequences are those in which J is a member selected from the numbers from 4 to 8.

Many processes have been devised for the synthesis of libraries of peptides and peptide analogs, which are applicable to practicing the present invention (see, for example, Gordon and Kerwin, COMBINATORIAL CHEMISTRY AND MOLECULAR DIVERSITY IN DRUG DISCOVERY, Wiley-Liss, New York, 1998).

Libraries of peptides and certain types of peptide mimetics, called “peptoids”, have been assembled and screened for a desirable biological activity by a range of methodologies (see, Gordon et al., J. Med Chem., 37: 1385-1401 (1994). For example, the method of Geysen, (Bioorg. Med. Chem. Letters, 3: 397-404 (1993); Proc. Natl. Acad Sci. USA, 81: 3998 (1984)) employs a modification of Merrifield peptide synthesis, wherein the C-terminal amino acid residues of the peptides to be synthesized are linked to solid-support particles shaped as polyethylene pins; these pins are treated individually or collectively in sequence to introduce additional amino-acid residues forming the desired peptides. The peptides are then screened for activity without removing them from the pins. The solid support of the invention can be similarly formed and used as a solid support for the synthesis of peptide libraries or other libraries.

Houghton, Proc. Natl. Acad. Sci. USA, 82: 5131 (1985); Eichler et al., Biochemistry, 32: 11035-11041 (1993); and U.S. Pat. No. 4,631,211) utilize individual polyethylene bags (“tea bags”) containing C-terminal amino acids bound to a solid support. These are mixed and coupled with the requisite amino acids using solid phase synthesis techniques. The peptides produced are then recovered and tested individually.

Fodor et al., Science, 251: 767 (1991), describe light-directed, spatially addressable parallel-peptide synthesis on a silicon wafer to generate large arrays of addressable peptides that can be directly tested for binding to biological targets. The solid support of the invention can be utilized in a similar manner.

In another combinatorial approach, equally applicable to the present invention, Huebner et al. (U.S. Pat. No. 5,182,366) discloses functionalized polystyrene beads divided into portions, each of which is acylated with a desired amino acid; the bead portions are mixed together, then divided into portions each of which is re-subjected to acylation with a second amino acid producing dipeptides. By using this synthetic scheme, exponentially increasing numbers of peptides are produced in uniform amounts, which are then separately screened for a biological activity of interest.

Presently preferred uses for the peptide libraries of the invention include their use in probing the reactivity and substrate specificity of enzymes, and in particular proteases. Thus, preferred libraries are those in which at least one peptide sequence of the library is cleavable by a protease into a fluorescent moiety and the peptide sequence, or a fragment of the peptide sequence.

The present invention provides techniques for preparing and probing peptide libraries having a wide range of sizes. Thus, in a preferred embodiment, the library includes at least 10 peptides, wherein each of the peptide sequences is a different peptide sequence. More preferably, the library includes at least 100 peptides, wherein each of the peptide sequences is a different peptide sequence, more preferably at least 1,000 peptides, still more preferably, at least 10,000 peptides, more preferably, at least 100,000 peptides, and even still more preferably, at least 1,000,000 peptides.

In another preferred embodiment, the peptide library of the invention is provided with a means by which a library member (e.g., peptide sequence) can be resolved from the other library members. Many such means for deconvoluting a library of compounds are known in the art, including, for example, the use of tags, positional libraries, and ordered arrays. Thus, in a preferred embodiment, the peptide library of the invention has a first member located at a first region of a substrate and a second member located at a second region of a substrate.

Libraries in a positional or an ordered array motif are presently preferred. Such libraries permit the identification of peptides, or other compounds, that are associated with zones of activity located during screening the library. Specifically, the library can be ordered so that the position of the peptide on the array corresponds to the identity of the peptide. Thus, once an assay has been carried out, and the position on the array determined for an active peptide, the identity of that peptide can be easily ascertained.

In another preferred embodiment, the present invention provides a library in a microarray format comprising n compounds distributed over n regions of a substrate. Preferably, each of the n compounds is a different compound. In a still further preferred embodiment, the n compounds are patterned on the substrate in a manner that allows the identity of the compound at each of the n locations to be ascertained. The microarray is patterned from essentially any type of fluorogenic molecule of the invention, including, but not limited to, small organic molecules, peptides, nucleic acids, carbohydrates, antibodies, enzymes, and the like.

A variety of methods are currently available for making arrays of biological molecules, such as arrays of antibodies, nucleic acid molecules, peptides or proteins. The following discussion utilizes a DNA microarray as an exemplary microarray. This use of DNA is intended to be illustrative and not limiting. One of skill in the art will appreciate that the following discussion is substantially applicable to forming microarrays of other fluorogenic compounds of the invention as well.

One method for making ordered arrays of compounds on a porous membrane is a “dot blot” approach. In this method, a vacuum manifold transfers a plurality, e.g., 96, aqueous samples of a compound from 3 millimeter diameter wells to a porous membrane. A common variant of this procedure is a “slot-blot” method in which the wells have highly-elongated oval shapes.

The compound is immobilized on the porous membrane by, for example, baking the membrane or exposing it to UV radiation. This is a manual procedure practical for making one array at a time and usually limited to 96 samples per array.

A more efficient technique employed for making ordered arrays of compounds uses n array of pins dipped into the wells, e.g., the 96 wells of a microtitre plate, for transferring an array of samples to a substrate, such as a porous membrane. One array includes pins that are designed to spot a membrane in a staggered fashion, for creating an array of 9216 spots in a 22×22 cm area. See, Lehrach, et al., HYBRIDIZATION FINGERPRINTING IN GENOME MAPPING AND SEQUENCING, GENOME ANALYSIS, Vol. 1, Davies et al, Eds., Cold Springs Harbor Press, pp. 39-81 (1990).

An alternate method of creating ordered arrays of compounds is described by Pirrung et al. (U.S. Pat. No. 5,143,854, issued 1992), and also by Fodor et al., (Science, 251: 767-773 (1991)) for preparing arrays of nucleic acid sequences. The method involves synthesizing different compounds at different discrete regions of a substrate. A related method has been described by Southern et al. (Genomics, 13: 1008-1017 (1992)).

Khrapko, et al., DNA Sequence, 1: 375-388 (1991) describes a method of making a compound matrix by spotting DNA onto a thin layer of polyacrylamide. The spotting is done manually with a micropipette.

When the library is associated with a substrate, the substrate can also be patterned using techniques such as photolithography (Kleinfield et al., J. Neurosci. 8:4098-120 (1998)), photoetching, chemical etching and microcontact printing (Kumar et al., Langmuir 10:1498-511 (1994)). Other techniques for forming patterns on a substrate will be readily apparent to those of skill in the art.

The size and complexity of the pattern on the substrate is limited only by the resolution of the technique utilized and the purpose for which the pattern is intended. For example, using microcontact printing, features as small as 200 nm are layered onto a substrate. See, Xia, Y.; Whitesides, G., J. Am. Chem. Soc. 117:3274-75 (1995). Similarly, using photolithography, patterns with features as small as 1 μm have been produced. See, Hickman et al., J. Vac. Sci. Technol. 12:607-16 (1994).

The pattern can be printed directly onto the substrate or, alternatively, a “lift off” technique can be utilized. In the lift off technique, a patterned resist is laid onto the substrate, a compound is laid down in those areas not covered by the resist and the resist is subsequently removed. Resists appropriate for use with the substrates of the present invention are known to those of skill in the art. See, for example, Kleinfield et al., J. Neurosci. 8:4098-120 (1998). Following removal of the photoresist, a second compound, having a structure different from the first compound can be bonded to the substrate on those areas initially covered by the resist. Using this technique, substrates with patterns having regions of different chemical characteristics can be produced. Thus, for example, a pattern having an array of adjacent wells can be created by varying the hydrophobicity/hydrophilicity, charge and other chemical characteristics of the pattern constituents. In one embodiment, hydrophilic compounds can be confined to individual wells by patterning walls using hydrophobic materials. Similar substrate configurations are accessible through microprinting a layer with the desired characteristics directly onto the substrate. See, Mrkish, M.; Whitesides, G. M., Ann. Rev. Biophys. Biomol. Struct. 25:55-78 (1996).

Structural Specificity Database

As high-resolution, high-sensitivity enzyme sequence specificity and datasets become available to the art, significant progress in the areas of diagnostics, therapeutics, drug development, biosensor development, and other related areas is possible. For example, disease markers can be identified and utilized for better confirmation of a disease condition or stage (see, U.S. Pat. Nos. 5,672,480; 5,599,677; 5,939,533; and 5,710,007). Subcellular toxicological information can be generated to better direct drug structure and activity correlation (see, Anderson, L., “Pharmaceutical Proteomics: Targets, Mechanism, and Function,” paper presented at the IBC Proteomics conference, Coronado, Calif. (Jun. 11- 12, 1998)). Subcellular toxicological information can also be utilized in a biological sensor device to predict the likely toxicological effect of chemical exposures and likely tolerable exposure thresholds (see, U.S. Pat. No. 5,811,231). Similar advantages accrue from datasets relevant to other biomolecules and bioactive agents (e.g., nucleic acids, saccharides, lipids, drugs, and the like).

Thus, in another preferred embodiment, the present invention provides a database that includes at least one set of the structural moiety (e.g., peptide sequence) specificity data for an enzyme, preferably a protease. The data contained in the database is acquired using a method of the invention and/or a detectable (e.g., fluorogenic) species of the invention either singly or in a library format. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases including structural (e.g., peptide sequence) specificity data is for clarity of illustration only. It will be apparent to those of skill in the art that similar databases can be assembled for any of the compounds or libraries of compounds of the present invention.

The compositions and methods described herein for identifying and/or quantifying the relative and/or absolute abundance of a variety of molecular and macromolecular species from a biological sample provide an abundance of information, which can be correlated with pathological conditions, predisposition to disease, drug testing, therapeutic monitoring, gene-disease causal linkages, identification of correlates of immunity and physiological status, among others. As the large amounts of raw data generated by these methods are poorly suited for manual review and analysis without prior data processing using high-speed computers, several methods for indexing and retrieving biomolecular information have been proposed. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

The present invention provides a method for producing a computer database comprising a computer and software for storing in computer-retrievable form a collection of enzyme peptide sequence specificity records cross-tabulated, for example, with data specifying the source of the protein-containing sample from which each sequence specificity record was obtained.

In a preferred embodiment, at least one of the sources of protein-containing sample is from a tissue sample known to be free of pathological disorders. In a variation, at least one of the sources is a known pathological tissue specimen, for example, a neoplastic lesion or a tissue specimen containing an infectious agent such as a virus, or the like. In another variation, the sequence specificity records cross-tabulate one or more of the following parameters for each protein species in a sample: (1) a unique identification code, which can comprise a peptide sequence specificity and/or characteristic separation coordinate (e.g., electrophoretic coordinates); (2) sample source; (3) absolute and/or relative quantity of the protein species present in the sample; (4) presence or absence of amine- or carboxy-terminal post-translational modifications; and (5) original amino acid sequence, electrophoresis and/or mass spectral data, and the like, used to identify the proteins.

The invention also provides for the storage and retrieval of a collection of peptide sequence specificities in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the peptide sequence specificity records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 protein species cross-tabulated with sample source.

The invention preferably provides a method for identifying related peptide sequences, comprising performing a computerized comparison between a peptide sequence specificity stored in or retrieved from a computer storage device or database and at least one other sequence; such comparison can comprise a sequence analysis or comparison algorithm or computer program embodiment thereof (e.g., FASTA, TFASTA, GAP, BESTFIT) and/or the comparison may be of the relative amount of a peptide sequence in a pool of sequences determined from a polypeptide sample of a specimen. The invention provides a computer system comprising a storage device having a bit pattern encoding a database having at least 100 protein expression fingerprint records obtained by the methods of the invention, and a program for sequence alignment and comparison to predetermined genetic or protein sequences.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding a protein expression fingerprint record; often the disk will comprise at least one other bit pattern encoding a polynucleotide and/or polypeptide sequence other than a peptide sequence record of the invention, typically in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal tranmission medium, whereby at least one network device (e.g., computer, disk array, etc.) comprises a pattern of magnetic domains (e.g., magnetic disk) and/or charge domains (e.g., an array of DRAM cells) composing a bit pattern encoding a protein expression fingerprint record of the invention.

The invention also provides a method for transmitting a structural (e.g., peptide sequence) specificity record of the invention that includes generating an electronic signal on an electronic communications device, such as a modem, ISDN terminal adapter, DSL, cable modem, ATM switch, or the like, wherein the signal includes (in native or encrypted format) a bit pattern encoding a peptide sequence specificity record or a database comprising a plurality of peptide sequence specificity records obtained by the method of the invention.

In a preferred embodiment, the invention provides a computer system for comparing a query polypeptide sequence or query peptide sequence specificity to a database containing an array of data structures, such as a peptide sequence specificity record obtained by the method of the invention, and ranking database sequences based on the degree of sequence identity and gap weight to the query sequence. A central processor is initialized to load and execute the computer program for alignment and/or comparison of the amino acid sequences. A query sequence including at least 2 amino acids or 6 nucleotides encoding 2 amino acids is entered into the central processor via an I/O device. Execution of the computer program results in the central processor retrieving the sequence data from the data file, which comprises a binary description of a peptide sequence specificity record or portion thereof containing polypeptide sequence data for the record.

The sequence data or record and the computer program can be transferred to secondary memory, which is typically random access memory (e.g., DRAM, SRAM, SGRAM, or SDRAM). Sequences are ranked according to the degree of sequence identity to the query sequence and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

In another preferred embodiment, the invention provides a computer program for comparing query polypeptide sequence(s) or query polynucleotide sequence(s) to a peptide sequence specificity database obtained by a method of the invention and ranking database sequences based on the degree of similarity of protein species expressed and relative and/or absolute abundance in a sample. The initial step is input of a query peptide sequence, or peptide sequence specificity record obtained by a method of the invention, input via an I/O device. A data file is accessed in to retrieve a collection of peptide sequence specificity records for comparison to the query. Individually or collectively sequences or other cross-tabulated information of the peptide sequence specificity collection are optimally matched to the query sequence(s), such as by the algorithm of Needleman and Wunsch or the algorithm of Smith and Waterman or another suitable algorithm obtainable by those skilled in the art.

Once aligned or matched, the percentage of sequence similarity can be computed for each aligned or matched sequence to generate a similarity value for each sequence or peptide sequence specificity record collection as compared to the query sequence(s). Sequences are generally ranked in order of greatest sequence identity or weighted match to the query sequence, and the relative ranking of the sequence to the best matches in the collection of records is thus generated. A determination is made; if more sequences records exist in the data file, the additional sequences or a subset thereof are retrieved and the process is iterated. If no additional sequences exist in the data file, the rank ordered sequences are output via an I/O device, thereby displaying the relative ranking of sequences among the sequences of the data file optimally matched and compared to the query sequence(s).

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison sequence, such as a query sequence; and (4) a program for alignment and comparison, typically with rank-ordering of comparison results on the basis of computed similarity values.

In a preferred embodiment, neural network pattern matching/recognition software is trained to identify and match structural (e.g., peptide sequence) specificity records based on backpropagation using empirical data input by a user. The computer system and methods described herein permit the identification of the relative relationship of a query structural (e.g., peptide sequence) specificity to a collection of structural (e.g., peptide sequence) specificities; preferably peptide sequence specificities (query and database) are obtained by the methods of the invention.

The invention also provides a computer system including a database containing a plurality of structural (e.g., peptide sequence) specificity records in the form of tree-based or otherwise hierarchical navigational fields cross-tabulated to informational data such as one or more or the following: medical records, patient medical history, medical diagnostic test results of a patient, patient name, patient sex, patient age, patient genetic profile, patient diagnosis-related group code, patient therapy, time of day, vital signs of a patient, drug assay results of a patient, medical information of patient's blood relatives, and other similar medical, biological, and physiological information of a patient from which the sample(s) used to generate the structural (e.g., peptide sequence) specificity record was obtained.

In a preferred embodiment, a computer system comprising a database having a hybrid data structure with the navigational field(s) comprising a peptide sequence specificity obtained by a method of the invention is employed to link to informational fields of the same or a related record which comprise medical information as described herein; the data structure can conform to the general description in U.S. Pat. No. 5,295,261, which is incorporated herein by reference.

As another example, the invention also provides a computer system, including a computer and a program employing a neural network trained to extract database records having a predicted or predetermined peptide sequence specificity match that is pathognomonic for a predetermined disease or medical condition, predisposition to disease, or physiological state. In an illustrative embodiment, a blood or cellular sample from a patient is analyzed according to a method of the invention to provide a predetermined peptide sequence specificity that is entered as a database query into a trained neural network that has been previously trained on a plurality of predetermined database records to establish correlative neural relationships between peptide sequence specificity (navigation fields) and medical data (information field(s)), so that the query identifies the medical condition(s) most highly correlated in the trained neural network with a peptide sequence specificity. The method can alternatively, or in addition, employ a predetermined peptide sequence specificity record obtained from serum, blood, or other cellular sample to query a database of sequence specificity profile records using a trained neural network which links the query metabolite profile record to the database records linked to the medical condition(s) most highly correlated in the trained neural network with the patient's peptide sequence specificity.

The invention also provides, for instance, a computer system, including a computer and a program employing a database comprising records having a field or plurality of fields including, for example, a peptide sequence specificity data set obtained from a serum, blood, or other cellular sample of a patient and analyzed according to a method of the present invention, and further having one or a plurality of fields containing data obtained from a patient relating to symptoms, medical status, medical history, or other differential diagnosis information, which can be entered via a connection to the Internet or other TCP/IP or related networking system.

Kits

The present invention also provides for kits for the detection of a selected species e.g., enzyme, nucleic acid, etc.) or activity (e.g., enzymatic, hybridization, etc.) in samples. The kits comprise one or more containers containing the compounds (“indicators”) of the present invention. The compounds may be provided in solution or bound to a solid support. Thus, the kits may contain indicator solutions or indicator “dipsticks”, blotters, culture media, and the like. The kits may also contain indicator cartridges (where the compound is bound to a solid support) for use in automated protease activity detectors.

The kits additionally may include an instruction manual that teaches a method of the invention and describes the use of the components of the kit. In addition, the kits may also include other reagents, buffers, various concentrations of enzyme inhibitors, stock enzymes (for generation of standard curves, etc), culture media, disposable cuvettes and the like to aid the detection of enzymatic activity utilizing the indicator compounds of the present invention.

It will be appreciated that kits may additionally, or alternatively, include any of the other indicators described herein (e.g., nucleic acid based indicators, oligosaccharide indicators, lipid indicators, etc.).

In another embodiment, the kit contains a solid support of the invention and, optionally, directions for using the solid support for preparing a compound of the invention. The kit may also contain reagents, buffers, etc. useful in preparing a detectable moiety conjugate of the invention.

Methods

Protease Assay

The assays of the invention are illustrated by the following discussion focusing on protease assays. The focus of this discussion is for clarity of illustration and should not be interpreted as limiting the scope of the invention to assays of proteases. Those of skill in the art will appreciate that the broad range of compounds that can be produced using the present invention can be assayed using methods known in the art or modifications on those methods that are well within the abilities of one of skill in the art.

Proteases represent a number of families of proteolytic enzymes that catalytically hydrolyze peptide bonds. Principal groups of proteases include metalloproteases, serine proteases, cysteine proteases and aspartic proteases. Proteases, in particular serine proteases, are involved in a number of physiological processes such as blood coagulation, fertilization, inflammation, hormone production, the immune response and fibrinolysis.

Numerous disease states are caused by and can be characterized by alterations in the activity of specific proteases and their inhibitors. For example emphysema, arthritis, thrombosis, cancer metastasis and some forms of hemophilia result from the lack of regulation of serine protease activities (see, for example, TEXTBOOK OF BIOCHEMISTRY WITH CLINICAL CORRELATIONS, John Wiley and Sons, Inc. N.Y. (1993)). In case of viral infection, the presence of viral proteases have been identified in infected cells. Such viral proteases include, for example, HIV protease associated with AIDS and NS3 protease associated with Hepatitis C. These viral proteases play a critical role in the virus life cycle.

Proteases have also been implicated in cancer metastasis. Increased synthesis of the protease urokinase has been correlated with an increased ability to metastasize in many cancers. Urokinase activates plasmin from plasminogen which is ubiquitously located in the extracellular space and its activation can cause the degradation of the proteins in the extracellular matrix through which the metastasizing tumor cells invade. Plasmin can also activate the collagenases thus promoting the degradation of the collagen in the basement membrane surrounding the capillaries and lymph system thereby allowing tumor cells to invade into the target tissues (Dano, et al. Adv. Cancer. Res., 44: 139 (1985)).

Human mast cells express at least four distinct tryptases, designated αβI, βII, and βIII. These enzymes are not controlled by blood plasma proteinase inhibitors and only cleave a few physiological substrates in vitro. The tryptase family of serine proteases has been implicated in a variety of allergic and inflammatory diseases involving mast cells because of elevated tryptase levels found in biological fluids from patients with these disorders. However, the exact role of tryptase in the pathophysiology of disease remains to be delineated. The scope of biological functions and corresponding physiological consequences of tryptase are substantially defined by their substrate specificity.

Tryptase is a potent activator of pro-urokinase plasminogen activator (uPA), the zymogen form of a protease associated with tumor metastasis and invasion. Activation of the plasminogen cascade, resulting in the destruction of extracellular matrix for cellular extravasation and migration, may be a function of tryptase activation of pro-urokinase plasminogen activator at the P4-P1′sequence of Pro-Arg-Phe-Lys (Stack, et al., Journal of Biological Chemistry 269(13): 9416-9419 (1994)). Vasoactive intestinal peptide, a neuropeptide that is implicated in the regulation of vascular permeability, is also cleaved by tryptase, primarily at the Thr-Arg-Leu-Arg sequence (Tam, et al., Am. J. Respir. Cell Mol. Biol. 3: 27-32 (1990)). The G-protein coupled receptor PAR-2 can be cleaved and activated by tryptase at the Ser-Lys-Gly-Arg sequence to drive fibroblast proliferation, whereas the thrombin activated receptor PAR-1 is inactivated by tryptase at the Pro-Asn-Asp-Lys sequence (Molino et al., Journal of Biological Chemistry 272(7): 4043-4049 (1997)). Taken together, this evidence suggests a central role for tryptase in tissue remodeling as a consequence of disease. This is consistent with the profound changes observed in several mast cell-mediated disorders. One hallmark of chronic asthma and other long-term respiratory diseases is fibrosis and thickening of the underlying tissues that could be the result of tryptase activation of its physiological targets. Similarly, a series of reports during the past year have shown angiogenesis to be associated with mast cell density, tryptase activity and poor prognosis in a variety of cancers (Coussens et al., Genes and Development 13(11): 1382-97 (1999)); Takanami et al., Cancer 88(12): 2686-92 (2000); Toth-Jakatics et al., Human Pathology 31(8): 955-960 (2000); Ribatti et al., International Journal of Cancer 85(2): 171-5 (2000)).

Tryptase has been recognized as a viable drug target, and therapeutically useful inhibitors have been under development by several pharmaceutical companies, some even taking advantage of the bifunctional active site (Burgess et al., Proceedings of the National Academy of Sciences 96(15): 8348-52 (1999); Rice et al., Curr Pharm Des 4(5): 381-96 (1998)). Insights gained from the modeling of the optimal sequence into the active site will support further development of novel selective substrates of β-tryptases that will enhance our understanding of the pathophysiology of these enzymes, as well as lead to the development of new and effective inhibitors.

Clearly, measurement of changes in the activity of specific proteases is clinically significant in the treatment and management of the underlying disease states. Proteases, however, are not easy to assay. Typical approaches include ELISA using antibodies that bind the protease or RIA using various labeled substrates; with their natural substrates assays are difficult to perform and expensive. With currently available synthetic substrates the assays are expensive, insensitive and nonselective. In addition, many “indicator” substrates require high quantities of protease which results, in part, in the self destruction of the protease.

Thus, in a preferred embodiment, the invention provides a method of assaying for the presence of an enzymatically active protease in a sample. The method includes contacting the sample with a peptide-detectable moiety conjugate of the invention, in such a manner that the detectable moiety is released from the peptide sequence upon action of the protease, thereby releasing the detectabel moiety from the peptide. The detectable moiety complexes a metal ion (or a metal chelate) and the sample is observed to determine whether it undergoes a detectable change in fluorescence. The detectable change is an indication of the presence of the enzymatically active protease in the sample.

The method of the invention can be used to assay for substantially any known or later discovered enzyme and is of particular use in assaying for a protease. The sample containing the protease can be derived from substantially any source, or organism. In a preferred embodiment, the sample is a clinical sample from a subject. In a presently preferred embodiment, the protease is a member selected from the group consisting of aspartic protease, cysteine protease, metalloprotease and serine protease. The method of the invention is particularly preferred for the assay of proteases derived from a microorganism, including, but not limited to, bacteria, fungi, yeast, viruses, and protozoa.

In an illustrative application, the compounds of this invention are used to assay the activity of purified protease made up as a reagent (e.g. in a buffer solution) for experimental or industrial use. Like many other enzymes, proteases may lose activity over time, especially when they are stored as their active forms. In addition, many proteases exist naturally in an inactive precursor form (e.g. a zymogen), which itself must be activated by hydrolysis of a particular peptide bond to produce the active form of the enzyme prior to use. Because the degree of activation is variable and because proteases may loose activity over time, it is often desirable to verify that the protease is active and to often quantify the activity before using a particular protease in a particular application.

Assaying for protease activity of a stock solution simply relies upon adding a quantity of the stock solution to a compound of the present invention and measuring the subsequent changes in a detectable signal, e.g., increase in fluorescence or decrease in excitation band in the absorption spectrum. The stock solution and the compound may also be combined and assayed in a “digestion buffer” that optimizes activity of the protease. Buffers suitable for assaying protease activity are well known to those of skill in the art. In general, a buffer will be selected whose pH corresponds to the pH optimum of the particular protease.

The fluorescence measurement is most easily made in a fluorometer, and instrument that provides an “excitation” light source for the fluorophore and then measures the light subsequently emitted at a particular wavelength. Comparison with a control indicator solution lacking the protease provides a measure of the protease activity. The activity level may be precisely quantified by generating a standard curve for the protease/indicator combination in which the rate of change in fluorescence produced by protease solutions of known activity is determined.

While detection of the fluorogenic compounds is preferably accomplished using a fluorometer, detection may be accomplished by a variety of other methods well known to those of skill in the art. Thus, for example, since the fluorophores of the present invention emit in the visible wavelengths, detection may be simply by visual inspection of fluorescence in response to excitation by a light source. Detection may also be by means of an image analysis system utilizing a video camera interfaced to a digitizer or other image acquisition system. Detection may also be by visualization through a filter, as under a fluorescence microscope. The microscope may provide a signal that is simply visualized by the operator. Alternatively, the signal may be recorded on photographic film or using a video analysis system. The signal may also simply be quantified in real time using either an image analysis system or a photometer.

Thus, for example, a basic assay for protease activity of a sample will involve suspending or dissolving the sample in a buffer (at the pH optima of the particular protease being assayed), adding to the buffer one of the compounds of the present invention, and monitoring the resulting change in fluorescence using a spectrofluorometer. The spectrofluorometer will be set to excite the fluorophore at the excitation wavelength of the fluorophore and to detect the resulting fluorescence at the emission wavelength of the fluorophore.

Previous approaches to verifying or quantifying protease activity involve combining an aliquot of the protease with its substrate, allowing a period of time for digestion to occur and then measuring the amount of digested protein, most typically by HPLC. This approach is time consuming, utilizes expensive reagents, requires a number of steps and entails a considerable amount of labor. In contrast, the fluorogenic reagents of the present invention allow rapid determination of protease activity in a matter of minutes in a single-step procedure. An aliquot of the protease to be tested is simply added to, or contacted with, the fluorogenic reagents of this invention and the subsequent change in fluorescence is monitored (e.g., using a fluorometer or a fluorescence microplate reader).

Moreover, the methods of the invention allow for the detection of alterations in fluorescence intensity in real time.

In addition to determining protease activity in “reagent” solutions, the compositions of the present invention may be utilized to detect protease activity in biological samples. The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

In one embodiment, the present invention provides for methods of detecting protease activity in an isolated biological sample. This may be determined by simply contacting the sample with a compound of the present invention and monitoring the change in fluorescence over time. The sample may be suspended in a “digestion buffer” as described above. The sample may also be cleared of cellular debris, e.g. by centrifugation before analysis.

In another embodiment, this invention provides for a method of detecting in situ protease activity in histological sections. This method of detecting protease activity in tissues offers significant advantages over prior art methods (e.g. specific stains, antibody labels, etc.) because, unlike simple labeling approaches, in situ assays using the protease indicators indicate actual activity rather than simple presence or absence of the protease. Proteases are often present in tissues in their inactive precursor (zymogen) forms which are capable of binding protease labels. Thus, traditional labeling approaches provide no information regarding the physiological state, vis a vis protease activity, of the tissue.

The in situ assay method generally comprises providing a tissue section (preferably a frozen section, as fixation or embedding may destroy protease activity in the sample), contacting the section with one of the compounds of the present invention, and visualizing the resulting fluorescence. Visualization is preferably accomplished utilizing a fluorescence microscope. The fluorescence microscope provides an “excitation” light source to induce fluorescence of the fluorophore. The microscope is typically equipped with filters to optimize detection of the resulting fluorescence. As indicated above, the microscope may be equipped with a camera, photometer, or image acquisition system.

The detectable moiety-peptide conjugate of the present invention can be introduced to the sections in a number of ways. For example, the compound may be provided in a buffer solution, as described above, which is applied to the tissue section. Alternatively, the compound may be provided as a semi-solid medium such as a gel or agar which is spread over the tissue sample. The gel helps to hold moisture in the sample while providing a signal in response to protease activity. The compound may also be provided conjugated to a polymer such as a plastic film which may be used in procedures similar to the development of Western Blots. The plastic film is placed over the tissue sample on the slide and the fluorescence resulting from cleaved indicator molecules is viewed in the sample tissue under a microscope.

Typically the tissue sample is incubated for a period of time sufficient to allow a protease to cleave the detectable moiety from the peptide. Incubation times will generally range from about 10 to 60 minutes at temperatures up to and including 37° C.

In yet another embodiment, this invention provides for a method of detecting in situ enzymatic activity of cells in culture or cell suspensions derived from tissues, biopsy samples, or biological fluids (e.g., saliva, blood, urine, lymph, plasma, etc.). In an illustrative embodiment, the cultured cells are grown either on chamber slides or in suspension and then transferred to histology slides by cytocentrifugation. Similarly, the cell suspensions are prepared according to standard methods and transferred to histology slides. The slide is washed with phosphate buffered saline and coated with a semi-solid polymer or a solution containing the detectable moiety-peptide compound. The slide is incubated at 37° C. for a time sufficient for a protease to cleave the compound. The slide is then examined under a fluorescence microscope equipped with the appropriate filters, as described above.

Alternatively, the cells are incubated with the compound at 37° C., then washed with buffer and transferred to a glass capillary tube and examined under a fluorescence microscope. When a flow cytometer is used to quantify the intracellular enzyme activity, the cells with the compound is simply diluted with buffer after 37° C. incubation and analyzed.

Previously described fluorogenic protease indicators typically absorb and emit light in the ultraviolet range (e.g., Wang et al., Tetrahedron Lett. 31:6493 (1990)). They are thus unsuitable for sensitive detection of protease activity in biological samples which typically contain constituents (e.g., proteins) that also absorb and emit in the ultraviolet range. In contrast, the fluorescent indicators of the present invention absorb in the ultraviolet range but emit in the visible range (400 nm to about 750 nm). These signals are, therefore, not readily quenched by, or otherwise interfered with by background molecules; therefore, they are easily detected in biological samples.

In an illustrative embodiment, the invention provides a library useful for profiling of various serine and cysteine proteases. The library is able to distinguish proteases having specificity for P1′-acidic amino acids, P1′-large hydrophobic, P1′-small hydrophobic, P1′-basic amino acids and P1′-multiple amino acids.

In another illustrative embodiment, the invention provides a library for probing the extended substrate specificity of various proteases, in which the P1′ position is held constant as any naturally occurring or synthetic amino acid, depending on the preferred P1′-specificity of the protease.

The invention also provides a library for probing the extended substrate specificity of proteases requiring a specific amino acid at any other position. For example, having P1′-positioned libraries including peptides having hydrophobic amino acids in the P2′ position.

The PS-SCL strategy provided by the present invention allows for the rapid and facile determination of proteolytic substrate specificity. Those of skill in the art will appreciate that the present invention provides a wide variety of alternative library formats. Determination and consideration of particular limitations relevant to any particular enzyme or method of substrate specificity determination are within the ability of those of skill in the art.

In addition to its use in assaying for the presence of a selected enzyme, the method of the invention is also useful for detecting, identifying and quantifying an enzyme (e.g., protease). Thus, in another preferred embodiment, the method further includes, (c) quantifying the fluorescent moiety, thereby quantifying the protease.

In yet another preferred embodiment, the invention provides a method of assaying for the presence of an enzyme, for example, an enzymatically active protease in a sample using a peptide of the invention. The method includes: (a) contacting the sample with the compound of the present invention, in such a manner whereby the detectable moiety is released from the peptide sequence upon action of the protease. The detectable moiety is complexed with a metal ion and the sample is observed to determine whether the sample undergoes a detectable change in fluorescence, the detectable change being an indication of the presence of the enzymatically active protease in the sample.

In a preferred embodiment of the above-described method, the method further includes, (d) quantifying the change in the detectable signal, thereby quantifying the enzyme.

In yet another preferred embodiment, the invention provides a method of assaying for the presence of any hydrolytic enzyme, for example, an enzymatically active esterase in a sample. The method includes the step of contacting the sample with the compound of the present invention, which comprises a detectable moiety linked through an ester linkage to a structural moiety that provides the substrate specificity preferred by the enzyme of interest, which may include but are not limited to one or more specific amino acid, nucleic acid, or saccharide residue at certain position of the oligomer of the structural moiety. Such substrate specificity for this pre-determined enzyme would be well understood by one skilled in the art. The sample would then be contacted with the enzyme and enzyme activity would be measured as described above.

In an additional preferred embodiment, the invention provides a method of assaying the activity of a hydrolytic enzyme using magnetic resonance as a detectable signal. This method includes contacting the sample containing the enzyme of interest with the compound(s) of the present invention in such a manner whereby the detectable moiety is released from the substrate upon action of the enzyme. The detectable moiety then binds to a gadolinium(III) ion, resulting in a change in the nuclear magnetic resonance signal (specifically, a change in the longitudinal relaxation rates) of the hydrogen nuclei from the water molecules in the vicinity of the gadolinium. For a general description of the technique, see, e.g., Chemical Reviews, 1999, volume 99 pages 2293-2352.

Protease Sequence Specificity Assay

In another preferred embodiment, the present invention provides a method of determining the sequence specificity of an enzyme, and preferably of an enzymatically active protease. The method includes contacting the protease with a library of peptides of the invention in such a manner whereby the detectable moiety is released from the peptide sequence. The detectable moiety is complexed with a metal ion, thereby converting the previously silent moiety into a complex imparting a detectable signal. When the signal is detected in a reaction involving a particular compound, the peptide sequence specificity profile of the protease is determined from the peptide sequence of that compound.

In a preferred embodiment of the above-described method, the method further includes quantifying the protease by quantifying the change in the detectable signal.

Microorganism Assay

In a further preferred embodiment, the invention provides a method of assaying for the presence of a selected microorganism in a sample by probing the sequence specificity of an enzyme or other molecule produced or utilized by the microorganism. In an illustrative embodiment, the enzyme is a protease, which mediates peptide cleavage by the microorganism of one or more peptides of the invention. The method includes contacting a sample suspected of containing the selected microorganism with a compound of the invention, wherein the peptide comprises a sequence that is selectively cleaved by a protease of the selected microorganism, thereby releasing the detectable moiety from the peptide sequence. The detectable moiety is complexed with a metal ion, thereby forming a complex imparting a detectable signal, e.g., a fluorescent signal. When the signal is detected, the presence of the selected microorganism in the sample is confirmed. The preferred embodiments of the present method are substantially similar to those described in conjunction with the protease assay, supra.

In yet another preferred embodiment, the invention provides a method of assaying for the presence of a selected microorganism in a sample by probing the sequence specificity of peptide cleavage by a protease of the microorganism using a detectable moiety-peptide conjugate of the invention. The method includes contacting a sample suspected of containing the selected microorganism with the compound, which comprises a peptide sequence that is selectively cleaved by a protease of a selected microorganism, thereby releasing the detectable moiety from the peptide sequence. The detectable moiety is complexed with a metal ion, thereby converting into a signal-imparting complex. When the signal is detected, the presence of the selected microorganism in the sample is confirmed.

In a preferred embodiment of the above-described method, the method further includes quantifying the protease, the microorganism, or both, by quantifying the detectable signal.

The above-described method is useful to determine whether an unknown microorganism contains an enzyme that acts on a compound of the invention to liberate a detectable moiety, and it may be include within or utilized in conjunction with a device in which identification of an unknown microorganism is made on the basis of its enzyme content (see, for example, Mize, U.S. Pat. No. 5,055,594).

The methods of the invention are also useful for determining the effect of an agent, such as an antimicrobial agent on a microorganism. Thus, the invention can, for example, take the form of a process for determining the minimum inhibitory concentration (MIC) of an antimicrobial substance with respect to a microorganism under study (e.g., a clinical septic isolate). In an illustrative embodiment, a microorganism is treated with an antimicrobial agent that inhibits or destroys an enzyme or other molecule necessary for the growth and/or reproduction of the organism. The effect of the antimicrobial agent on the microorganism is probed by contacting the microorganism with one or more of the compounds of the invention. A change in the ability of the enzyme of the microorganism to produce a detectable signal from the compound is indicative of the activity of the antimicrobial agent. The magnitude of the effect, can be ascertained by quantifying the signal and comparing it to a selected benchmark, such as the level of the signal arising from contacting the microorganism with a compound of the invention in the absence of an antimicrobial agent (see, for example, Carr et al, U.S. Pat. No. 5,064,756, and U.S. Pat. No. 5,079,144).

In the above-recited methods, the exposure to the detectable moiety-containing compound of the invention to the microorganisms lasts for a sufficient time to let the enzymatic reaction take place. The detectable signal of each sample is assessed (e.g., by a non-destructive instrumental fluorometric or fluoroscopic method, or by magnetic resonance imaging).

Moreover, in each of the aspects and embodiments set forth hereinabove, the protease can be substantially any protease of interest, but is preferably a member selected from the group consisting of aspartic protease, cysteine protease, metalloprotease and serine protease. The protease assayed using a method of the invention can be derived from substantially any organism, including, but not limited to mammals, birds, reptiles, insects, plants, fungi and the like. In a preferred embodiment, the protease is derived from a microorganism, including, but not limited to, bacteria, fungi, yeast, viruses, and protozoa.

Synthesis of the Compounds of the Invention

Those of skill in the art will recognize that many methods can be used to prepare the compounds and the libraries of the invention. The compounds, especially those having a peptide as the structural moiety, are typically prepared by solid phase synthesis. For instance, after the synthesis of the peptide is complete, the peptide-detectable moiety conjugate can be cleaved from the solid support or, alternatively, the conjugate can remain tethered to the solid support.

Solid phase peptide synthesis in which the C-terminal amino acid of the sequence is attached to an insoluble support followed by sequential addition of the remaining amino acids in the sequence is the preferred method for preparing the peptide backbone of the compounds of the present invention. Techniques for solid phase synthesis are described by Barany and Merrifield, Solid-Phase Peptide Synthesis; pp. 3-284 in The Peptides: Analysis, Synthesis, Biology. Vol. 2; SPECIAL METHODS IN PEPTIDE SYNTHESIS, Part A.,Gross and Meienhofer, eds. Academic press, N.Y., 1980; and Stewart et al., SOLID PHASE PEPTIDE SYNTHESIS, 2nd ed. Pierce Chem. Co., Rockford, Ill. (1984) which are incorporated herein by reference. Solid phase synthesis is most easily accomplished with commercially available peptide synthesizers utilizing Fmoc or t-BOC chemistry.

In a particularly preferred embodiment, peptide synthesis is performed using Fmoc synthesis chemistry. The side chains of Asp, Ser, Thr and Tyr are preferably protected using t-butyl and the side chain of Cys residue using S-trityl and S-t-butylthio, and Lys residues are preferably protected using t-Boc, Fmoc and 4-methyhtrityl for lysine residues. Appropriately protected amino acid reagents are commercially available or can be prepared using art-recognized methods. The use of multiple protecting groups allows selective deblocking and coupling of a fluorophore to any particular desired side chain. Thus, for example, t-Boc deprotection is accomplished using TFA in dichloromethane. Fmoc deprotection is accomplished using, for example, 20% (v/v) piperidine in DMF or N-methylpyrolidone, and 4-methyltrityl deprotection is accomplished using, for example, 1 to 5% (v/v) TFA in water 30 or 1% TFA and 5% triisopropylsilane in DCM. S-t-butylthio deprotection is accomplished using, for example, aqueous mercaptoethanol (10%). Removal of t-butyl, t-boc and S-trityl groups is accomplished using, for example, TFA:phenol:water:thioanisol:ethanedithiol (85:5:5:2.5:2.5), or TFA:phenol:water (95:5:5).

Diversity at any particular position or combination of positions is introduced by utilizing a mixture of at least two, preferably at least 6, more preferably at least 12 and more preferably still, at least 20, amino acids to grow the peptide chain. Thus, a member selected from the group consisting of pA¹, pA², pA³ and combinations thereof includes a mixture of protected amino acids differing in the identity of the amino acid portion of the protected amino acids. The mixtures of amino acids can include of any useful amount of a particular amino acid in combination with any useful amount of one or more different amino acids. In a presently preferred embodiment, the mixture is an isokinetic mixture of amino acids.

In another preferred embodiment, the detectable moiety is covalently linked to a structural moiety, which is an oligomer of amino acid, nucleic acid, saccharide residues, or combinations thereof, through an ester linkage. There are a number of art-recognized methods that can be used for synthesizing such an oligomer, and for covalently linking the structural and detectable moieties by an ester bond. The structural moiety consists of one or more amino acid, nucleic acid, or saccharide residue, or their combinations, and may be a homo- or hetero- oligomer of such residues. An ester, thioester, or phosphate ester linkage may serve as the appropriate linking group. Appropriate protecting groups recognized by those skilled in the art can be used during synthesis if necessary to prevent undesirable side products from forming.

The materials and methods of the present invention are further illustrated by the examples which follow. These examples are offered to illustrate, but not to limit the claimed invention.

### EXAMPLES

### Example 1

Fsa-enhanced terbium ion fluorescence has been reported as a discontinuous assay for alkaline phosphatase activity^{18,19} wherein the reaction mixture was made basic with NaOH before fluorescence was measured. Our initial investigations into the fluorescence of the [Tb(EDTA)(fsa)] conjugate provided confirmation that this technique could also be valuable in the detection of peptide substrate hydrolysis. The sensitivity of the [Tb(EDTA)(fsa)] assay was measured at both elevated pH and pH 8.0. The fsa detection limit was approximately 1 μM in both cases. The slight increase in fluorescence intensity at elevated pH was not enough to justify the use of a discontinuous assay in which the reaction is quenched by addition of NaOH before measuring fluorescence. Instead we chose to use a continuous assay in which the increasing fluorescence is monitored in real time as substrate is hydrolyzed and fsa is released into the [Tb(EDTA)]+containing reaction solution. At pH 8.0, maximum sensitivity was obtained with a [Tb(EDTA)]+concentration of 10 μM.

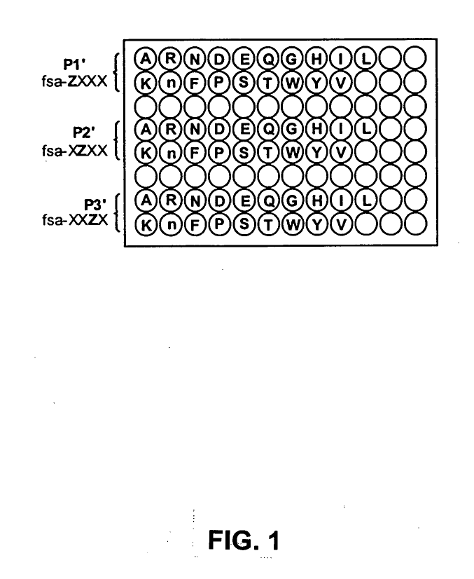

The fsa ligand was incorporated into a tetrapeptide library utilizing the positional scanning approach.²⁰ In this library, each substrate is a tetrapeptide with an amidated C-terminus and an fsa ligand attached to the N-terminus. Three sublibraries scanning the P1′, P2′ and P3′ positions were synthesized in 96-well plate format, as shown in FIG. 1. The P4′ position consists of an equimolar mixture^{21,22} of 19 amino acids—the 20 naturally occurring amino acids except methionine and cysteine but including the unnatural amino acid norleucine, a methionine isostere, were used in all cases. For example, in the P2′ sublibrary, the P2′ position is spatially addressed, with each of its 19 members contained in a separate well of the library plate.²³ The P1′, P3′ and P4′ positions are occupied by an equimolar mixture 22 of the 19 possible amino acids. By incorporating all 19 of the relevant amino acids at each of these positions, every possible tetrapeptide sequence is represented, facilitating a sampling of all sequence space.

The positionally scanned library was synthesized using standard solid phase amino acid coupling procedures as illustrated in Scheme 1. Rink amide resin was used as the solid support. The tetrapeptide chain was built upon the resin and the fsa was incorporated at the N-terminus using standard coupling protocols.⁵ The library was then cleaved from the resin under acidic conditions. Following the removal of solvent from the library, the residue was dissolved in DMSO to a final concentration of approximately 20 mM total substrate, estimated based on the approximately 40% yields obtained from similar single substrate syntheses. As each sublibrary has 3 randomized positions, there are 193, or 6859 different substrates in each library well and the concentration of each individual substrate approximately 2.9 μM. It is important to keep the concentration of substrate below the Kₘ so that the rate of hydrolysis remains directly proportional to the specificity constant, k_{cat}/Kₘ.

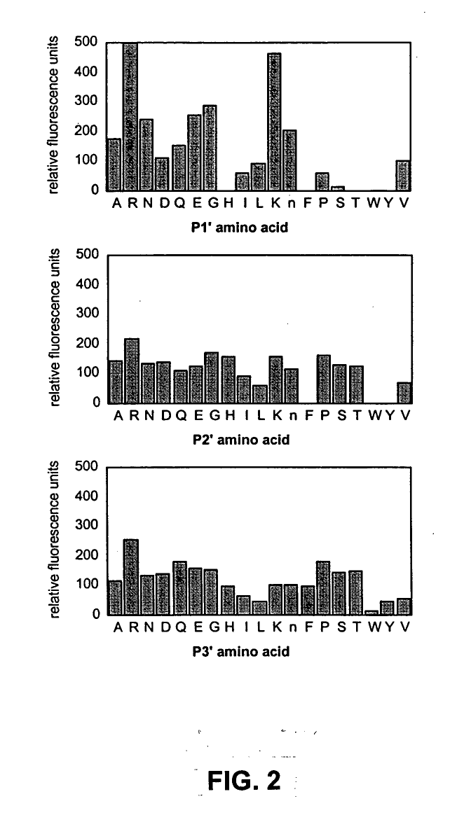

The combinatorial tetrapeptide library thus obtained was used to assay the substrate specificity of bovine p-chymotrypsin as follows. A 1 μL aliquot of each library member in DMSO was diluted to 100 μL in one well of a black polystyrene 96-well plate with a solution containing 50 mM Tris-HCl pH 8.0, 100 mM NaCl, 20 mM CaCl₂, 10 μM [Tb(EDTA)]+and 10 μM enzyme. The resulting mixture was allowed to react at 25° C. for 30 min. Hydrolysis was followed by observing the increase in fluorescence at 546 nm upon excitation at 250 nm. As shown in FIG. 2, the enzyme cleaves substrates with Arg and Lys in the P1′ position most efficiently. The P2′ position is more broad in its specificity and the P3′ position shows a slight preference for Arg.

The tetrapeptide library provides an averaged and independent view of the amino acid preferences at each subsite and does not address the possibility of cooperative effects between the subsites. To elucidate any cooperativity between subsites, a series of single substrates was synthesized and assayed with chymotrypsin. The individual substrates were synthesized in a similar fashion as the library and purified by reverse-phase HPLC. Peptide sequences were designed to compare the most reactive amino acid in each sublibrary (Arg) with an amino acid with moderate reactivity and neutral functionality (Ala). Alanine, a small, neutral amino acid, was included in the P4′ position in all peptides, providing extended backbone interactions to enhance turnover. The kinetic data obtained from single substrate hydrolysis were analyzed using the standard Michaelis-Menten equation. As outlined in Table 1, an arginine residue in the P1′ position is necessary for efficient turnover of the substrate. Of the single substrates listed in Table 1, the only substrate without a P1′ Arg residue for which satisfactory kinetic data could be obtained was fsa-ARRA, which had a k_{cat}/Kₘ an order of magnitude lower than the substrates with P1′ Arg. A detailed kinetic analysis of the hydrolysis of fsa-RRAA provided a kcat value of 2.2±0.2 s⁻¹ and a Kₘ value of 0.5±0.1 mM. Substrates with Arg at both P1′ and P3′ were hydrolyzed somewhat less efficiently than those with Arg at only P1′, supporting the proposal by Schellenberger et. al.²⁴ that both of these positively charged residues may interact with the same negatively charged residue(s) in the enzyme.

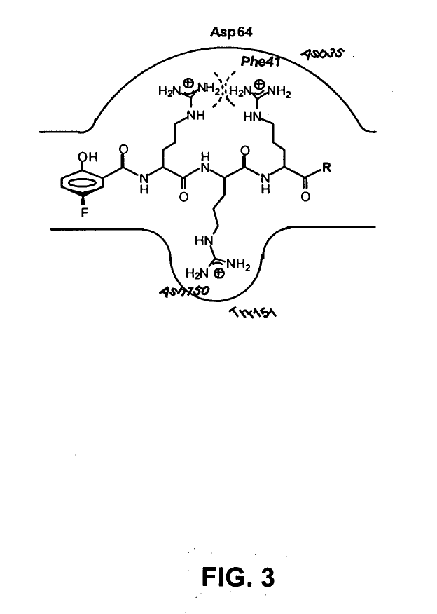

The prime site specificity for bovine α-chymotrypsin determined in these experiments matches quite well with that previously obtained using the acyl transfer method^{15,24,25} and can be interpreted based on the crystal structures of the enzyme complexed with macromolecular inhibitors as summarized in FIG. 3.²⁶ The residues Asp35 and Asp64 have both been predicted to interact with the P1′ and P3′ residues. Closer inspection of the crystal structure of bovine α-chymotrypsin complexed with turkey ovomucoid protein third domain reveals a possible cation-π interaction²⁷ (approximately 3.8-3.9 Å) between the P3′ Arg residue of the inhibitor and Phe41, providing another potentially favorable interaction with the positively charged residue.

The data from this study provide further evidence for the cooperativity between the S1′ and S3′ subsites in chymotrypsin,²⁴ confirming that single substrates with Arg in both the P1′ and P3′ positions are hydrolyzed less efficiently than those with only one Arg. In the S2′ subsite, a hydrogen bonding interaction between the amide NH group of the substrate and an oxygen from Phe41 of the enzyme has been the major determinant of specificity reported.²⁴ The library results show a relatively broad specificity in this site, but the single substrate kinetics data indicate that Arg may be slightly more favorable than Ala at P2′. Chymotrypsin does not seem to display a well-defined pocket for the P2′ residue of the substrate, but there are numerous opportunities for hydrogen bonding interactions between the P2′ guanidinium group and backbone and side chains from the enzyme, with Asn150 and Thr151 as two likely candidates.

### Example 2

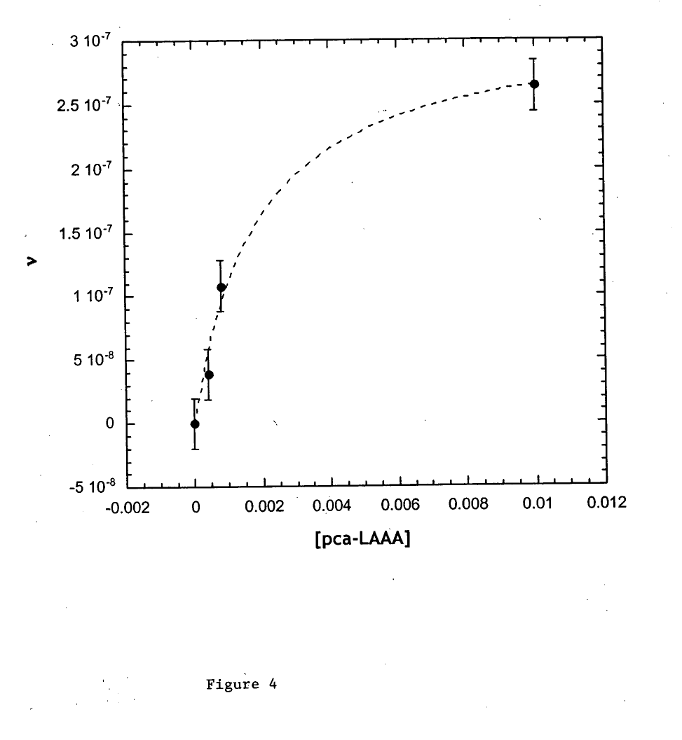

In another preferred embodiment, the detectable moiety of the compound of the present invention is phenanthroline carboxylic acid (pca). Experimental results indicate that pca can be linked to a peptide substrate through an amide linkage, and that cleavage of this linkage by a proteolytic enzyme can be measured in real time by following the increase in fluorescence due to the presence of a pca-Europium chelate that forms upon proteolysis. The data in FIG. 4 show hydrolysis of pca-LAAA by bovine alpha chymotrypsin. In this experiment, the enzyme concentration was held constant and the substrate concentration was varied as shown on the x-axis. The increase in pca-Europium fluorescence at 615 nm with exitation at 280 nm was followed. For this reactionFor this reaction, the k_{cat} is 9.7×10⁻⁷ s⁻¹ and K_{M} is 1.8 mM.

It is understood that the examples and embodiments described herein are for illustrative purposes only and that various modifications or changes in light thereof will be suggested to persons skilled in the art and are to included within the spirit and purview of this application and are considered within the scope of the appended claims. All patents, patent applications, and other publications cited herein are hereby incorporated by reference in their entirety for all purposes.

### REFERENCES AND NOTES

(1) Barrett, A. J.; Rawlings, N. D.; Woessner, J. F. Handbook of Proteolytic Enzymes; Academic Publishers: London, 1998.
(2) Harris, J. L.; Craik, C. S. Cell 2000, 136-137.
(3) Dunn, B. Nat. Biotechnol. 2000, 18, 149-150.
(4) Schechter, I.; Berger, A. Biochem. Biophys. Res. Commun. 1967, 27, 157-162.
(5) Backes, B. J.; Harris, J. L.; Leonetti, F.; Craik, C. S.; Ellman, J. A. Nat. Biotechnol. 2000, 18, 187-193.
(6) Harris, J. L.; Backes, B. J.; Leonetti, F.; Mahrus, S.; Ellman, J. A.; Craik, C. S. Proc. Natl. Acad. Sci. U S. A. 2000, 97, 7754-7759.
(7) Rano, T. A.; Timkey, T.; Peterson, E. P.; Rotonda, J.; Nicholson, D. W.; Becker, J. W.; Chapman, K. T.; Thomberry, N. A. Chemistry & Biology 1997, 4, 149-155.
(8) Thomberry, N. A.; Rano, T. A.; Peterson, E. P.; Rasper, D. M.; Timkey, T.; Garcia-Calvo, M.; Houtzager, V. M.; Nordstrom, P. A.; Roy, S.; Vaillancourt, J. P.; Chapman, K. T.; Nicholson, D. W. J. Biol. Chem. 1997, 272, 17907-17911.
(9) Takeuchi, T.; Shuman, M. A.; Craik, C. S. Proc. Natl. Acad. Sci. USA 1999, 96, 11054-11061.
(10) Dauber, D. S.; Ziermann, R.; Parkin, N.; Maly, D. J.; Mahrus, S.; Harris, J. L.; Ellman, J. A.; Petropoulos, C.; Craik, C. S. J. Virol. 2002, 76, 1359-1368.
(11) Harris, J. L.; Peterson, E. P.; Hudig, D.; Thormberry, N. A.; Craik, C. S. J. Biol. Chem. 1998, 273, 27364-27373.
(12) Matthews, D. J.; Wells, J. A. Science 1993, 260, 1113-1117.
(13) Krafft, G. A.; Wang, G. T. Methods Enzymol. 1994, 241, 70-86.
(14) Matayoshi, E. D.; Wang, G. T.; Kraffi, G. A.; Erickson, J. Science 1990, 247, 954-958.
(15) Schellenberger, V.; Turck, C. W.; Hedstrom, L.; Rutter, W. J. Biochemistry 1993, 32, 4349-4353.
(16) Coates, J.; Sammes, P. G.; West, R. M. J. Chem. Soc., Perkin Trans. 2 1996, 1275-1282.
(17) Coates, J.; Sammes, P. G.; West, R. M. J. Chem. Soc., Perkin Trans. 2 1996, 1283-1287.
(18) Christopoulos, T. K.; Diamandis, E. P. Anal. Chem. 1992, 64, 342-346.
(19) Evangelista, R. A.; Pollak, A.; Templeton, E. F. G. Anal. Biochem. 1991, 197, 213-224.
(20) Pinilla, C.; Appel, J. R.; Blanc, P.; Houghten, R. A. Biotechniques 1992, 13, 901-905.
(21) Houghten, R. A.; Pinilla, C.; Appel, J. R.; Blondelle, S. E.; Dooley, C. T.; Eichler, J.; Nefzi, A.; Ostresh, J. M. J. Med. Chem. 1999, 42, 3743-3778.
(22) Ostresh, J. M.; Winkle, J. H.; Hamashin, V. T.; Houghten, R. A. Biopolymers 1994, 34, 1681-1689.
(23) All 20 naturally occurring amino acids except methionine and cysteine were included in the library. Norleucine, an unnatural amino acid isosteric with methionine was also included.
(24) Schellenberger, V.; Turck, C. W.; Rutter, W. J. Biochemistry 1994, 33, 4251-4257.
(25) Schellenberger, V.; Schellenberger, U.; Mitin, Y. V.; Jakubke, J.-D. Eur. J. Biochem. 1990, 187, 163-167.
(26) Fujinaga, M.; Sielecki, A. R.; Read, R. J.; Ardelt, W.; Laskowski Jr., M.; James, M. N. G. J. Mol. Biol. 1987, 195, 397-418.

(27) Ma, J. C.; Dougherty, D. A. Chem. Rev. 1997, 97, 1303-1324.

TABLE 1
Single substrate kinetics with bovine α-chymotrypsin

| Substrate | k_{cat}/Kₘ (M⁻¹s⁻¹) |
|---|---|
| fsa-RRAA | 4400 ± 1000 |
| fsa-RAAA | 3500 ± 800 |
| fsa-RRRA | 2200 ± 500 |
| fsa-RARA | 1900 ± 500 |
| fsa-ARRA | 160 ± 60 |
| fsa-ARAA | NA |
| fsa-AARA | NA |

1. A compound comprising a detectable moiety linked by a covalent bond to a structural moiety, wherein upon cleavage of the covalent bond, the detectable moiety is capable of forming a complex with a lanthanide ion and the complex imparts a detectable signal, the compound having the formula:
R—X-A¹-A²-(Aⁱ)_{J-2} (I),
wherein:
R is the detectable moiety; A¹-A²-(Aⁱ)_{J-2} is the structural moiety consisting of an oligomer of amino acid, nucleotide, or saccharide residues; R is a substituted or unsubstituted aryl or substituted or unsubstituted heteroaryl moiety; X is a member selected from the group consisting of C(O)—NH, C(O)—O, and OP(O)(OH)—O; each of A¹ through Aⁱ is an amino acid, a nucleotide, or a saccharide residue;
J denotes the number of residues forming the homo-oligomer and is a member selected from the group consisting of the numbers from 2 to 10, such that J−2 is the number of residues in the oligomer sequence exclusive of A¹-A²; and
i denotes the position of the residue relevant to A¹ and when J is greater than 2, i is a member selected from the group consisting of the numbers from 3 to 10.

2. The compound of claim 1, which is attached to a solid support.

3. The compound of claim 1, which has the formula: 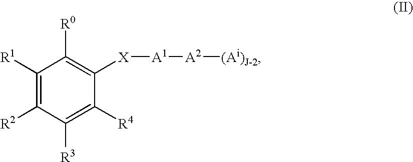
wherein:
R¹, R², R³, and R⁴ are independently selected from the group consisting of H, halogen, —NO₂, —CN, —C(O)ₘR⁵, —C(O)NR⁶R⁷, —S(O)ₜR′, —SO₂NR⁹R″, —OR″, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS;
R⁰, R⁵, R⁶, R⁷, R⁸, R⁹, R¹⁰, and R¹¹ are independently selected from the group consisting of H, halogen, substituted or unsubstituted alkyl, and substituted or unsubstituted heteroalkyl;
R¹² is either present or absent, and when present, is a member selected from the group consisting of substituted or unsubstituted alkyl, and substituted or unsubstituted heteroalkyl; when R¹² is absent, Y is attached directly to the detectable moiety;
R¹³ is a linking group adjoining the detectable moiety and the solid support;
m is a member selected from the group consisting of the integers from 1 to 2;
t is a member selected from the group consisting of the integers from 0 to 2;
Y is an organic functional group or methyl, and is a member selected from the group consisting of —COOR¹⁴, CONR¹⁴R¹⁵, —C(O)R¹⁴, —OR¹⁴, —SR¹⁴, N¹⁴R¹⁵, —C(O)NR¹⁴R¹⁵, and —C(O)SR¹⁴;
R¹⁴ and R¹⁵ are members independently selected from the group consisting of H, substituted or unsubstituted alkyl, substituted or unsubstituted heteroalkyl, substituted or unsubstituted aryl and substituted or unsubstituted heteroaryl; and
SS is a solid support.

4. The compound of claim 1, wherein X is C(O)—NH and each of the A¹ to Aⁱ is an amino acid residue independently selected from the group consisting of natural amino acids, unnatural amino acids, and modified amino acids.

5. The compound of claim 1, which has the formula:

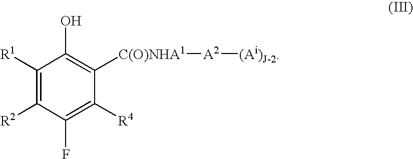

6. The compound of claim 1, which has the formula:
R—C(O)—NH-A (IV),
wherein:
A is an amino acid residue selected from the group consisting of natural amino acids, unnatural amino acids, and modified amino acids.

7. The compound of claim 1, which has the formula: 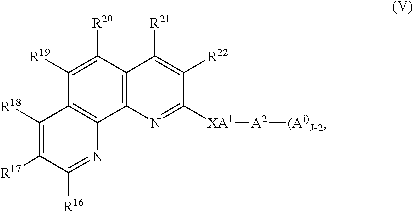
wherein:
R¹⁶ to R²² is independently selected from the group consisting of are independently selected from the group consisting of H, halogen, —NO₂, —CN, —C(O)ₘR⁵, —C(O)NR⁶R⁷, —S(O)ₜR⁸, —SO₂NR⁹R¹⁰, —OR¹¹, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS; and
R⁵, R⁶, R⁷, R⁸, R⁹, R¹⁰, and R¹¹ are independently selected from the group consisting of H, halogen, substituted or unsubstituted alkyl, and substituted or unsubstituted heteroalkyl.

8. The compound of claim 7, which has the formula:

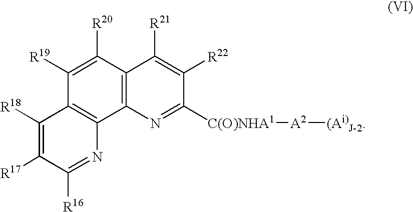

9. The compound of claim 1, wherein the lanthanide is terbium, europium, samarium, dysprosium, or gadolinium.

10. The compound of claim 1, wherein R is a 5-fluorosalicylic acid moiety.

11. The compound of claim 1, wherein R is a phenanthroline carboxylic acid moiety.

12. The compound of claim 1, wherein X is C(O)—O and each of A¹ to Aⁱ is a saccharide residue.

13. The compound of claim 1, wherein X is OP(O)(OH)—O and each of A¹ to Aⁱ is a nucleotide residue.

14. A library of compounds comprising at least a first and a second members, wherein each member is a compound comprising a detectable moiety linked by a covalent bond to a structural moiety, wherein upon cleavage of the covalent bond, the detectable moiety is capable of forming a complex with a lanthanide ion and the complex imparts a detectable signal, the compound having the structure:
R—X-A¹-A²-(Aⁱ)_{J-2} (I),
wherein:
R is the detectable moiety; A¹-A2-(Aⁱ)_{J-2} is the structural moiety consisting of an oligomer of amino acid, nucleotide, or saccharide residues; R is a substituted or unsubstituted aryl or substituted or unsubstituted heteroaryl moiety; X is a member selected from the group consisting of C(O)—NH, C(O)—O, and OP(O)(OH)—O; each of A¹ through Aⁱ is an amino acid, a nucleotide, or a saccharide residue;
J denotes the number of residues forming the oligomer and is a member selected from the group consisting of the numbers from 2 to 10, such that J−2 is the number of residues in the oligomer sequence exclusive of A¹-A²; and
i denotes the position of the residue relevant to A¹ and when J is greater than 2, i is a member selected from the group consisting of the numbers from 3 to 10.

15. The library of claim 14, wherein the compound has the formula: 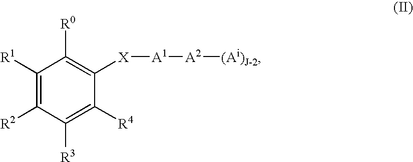
wherein:
R¹, R², R³, and R⁴ are independently selected from the group consisting of H, halogen, —NO₂, —CN, —C(O)ₙR⁵, —C(O)NR⁶R⁷, —S(O)ₜR⁸, —SO₂NR⁹R¹⁰, —OR¹¹, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS;
R⁰, R⁵, R⁶, R⁷, R⁸, R⁹, R¹⁰, and R¹¹ are independently selected from the group consisting of H, halogen, substituted or unsubstituted alkyl, and substituted or unsubstituted heteroalkyl;
R¹² is either present or absent, and when present, is a member selected from the group consisting of substituted or unsubstituted alkyl, and substituted or unsubstituted heteroalkyl; when R¹² is absent, Y is attached directly to the detectable moiety;
R¹³ is a linking group adjoining the detectable moiety and the solid support;
m is a member selected from the group consisting of the integers from 1 to 2;
t is a member selected from the group consisting of the integers from 0 to 2;
Y is an organic functional group or methyl, and is a member selected from the group consisting of —COOR¹⁴, CONR¹⁴R¹⁵, —C(O)R¹⁴, —OR¹⁴, —SR¹⁴, NR¹⁴R¹⁵, —C(O)NR¹⁴R¹⁵, and —C(O)SR¹⁴;
R¹⁴ and R¹⁵ are members independently selected from the group consisting of H, substituted or unsubstituted alkyl, substituted or unsubstituted heteroalkyl, substituted or unsubstituted aryl and substituted or unsubstituted heteroaryl; and
SS is a solid support.

16. The library of claim 14, wherein X is C(O)—NH and each of the A¹ to Aⁱ is an amino acid residue selected from the group consisting of natural amino acids, unnatural amino acids, and modified amino acids.

17. The library of claim 14, wherein the compound has the formula:

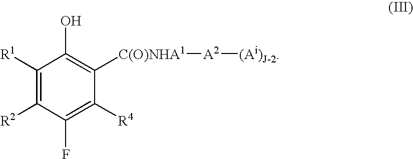

18. The library of claim 14, wherein the compound has the formula:
R—C(O)—NH-A (IV),
wherein:
A is an amino acid residue selected from the group consisting of natural amino acids, unnatural amino acids, and modified amino acids.

19. The library of claim 14, wherein the compound has the formula: 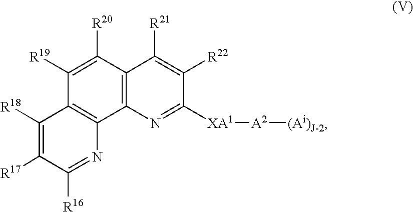
wherein:
R¹⁶ to R²² is independently selected from the group consisting of are independently selected from the group consisting of H, halogen, —NO₂, —CN, —C(O)ₘR⁵, —C(O)NR⁶R⁷, —S(O)ₜR⁸, —SO₂NR⁹R¹⁰, —OR¹¹, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS; and
R⁵, R⁶, R⁷, R⁸, R⁹, R¹⁰, and R¹¹ are independently selected from the group consisting of H, halogen, substituted or unsubstituted alkyl, and substituted or unsubstituted heteroalkyl.

20. The library of claim 19, wherein the compound has the formula:

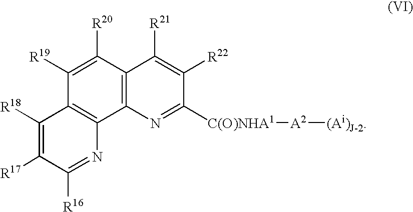

21. The library of claim 14, wherein each of the members is attached to a solid support.

22. The library of claim 14, wherein the members comprise at least two different detectable moieties.

23. The library of claim 14, wherein the lanthanide is terbium, europium, samarium, dysprosium, or gadolinium.

24. The library of claim 14, wherein the detectable moiety is a 5-fluorosalicylic acid moiety.

25. The library of claim 14, wherein the detectable moiety is a phenanthroline carboxylic acid moiety.

26. The library of claim 14, wherein the members are provided in an addressable array.

27. A method for identifying substrate specificity of an enzyme, comprising the steps of:
(a) contacting members of the library of claim 14 individually with the enzyme at the presence of an lanthanide ion under conditions permissible for the enzyme to cleave the covalent bond linking the detectable moiety and the structural moiety; and
(b) detecting change in fluorescence or magnetic resonance contrast, wherein an increase in fluorescence or magnetic resonance contrast indicates cleavage of the covalent bond, and thereby determining the substrate specificity of the enzyme from the structural moiety of the member.

28. The method of claim 27, wherein the compound has the formula: 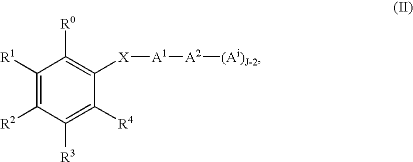
wherein:
R¹, R², R³, and R⁴ are independently selected from the group consisting of H, halogen, —NO₂, —CN, —C(O)ₘR⁵, —C(O)NR⁶R⁷, —S(O)ₜR⁸, —SO₂NR⁹R¹⁰, —OR¹¹, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS;
R⁰, R⁵, R⁶, R⁷, R⁸, R⁹, R¹⁰, and R¹¹ are independently selected from the group consisting of H, halogen, substituted or unsubstituted alkyl, and substituted or unsubstituted heteroalkyl;
R¹² is either present or absent, and when present, is a member selected from the group consisting of substituted or unsubstituted alkyl, and substituted or unsubstituted heteroalkyl; when R¹² is absent, Y is attached directly to the detectable moiety;
R¹³ is a linking group adjoining the detectable moiety and the solid support;
m is a member selected from the group consisting of the integers from 1 to 2;
t is a member selected from the group consisting of the integers from 0 to 2;
Y is an organic functional group or methyl, and is a member selected from the group consisting of —COOR¹⁴, CONR¹⁴R¹⁵, —C(O)R⁴, —OR¹⁴, —SR¹⁴, NR¹⁴R¹⁵, —C(O)NR¹⁴R¹⁵, and —C(O)SR¹⁴;
R¹⁴ and R¹⁵ are members independently selected from the group consisting of H, substituted or unsubstituted alkyl, substituted or unsubstituted heteroalkyl, substituted or unsubstituted aryl and substituted or unsubstituted heteroaryl; and
SS is a solid support.

29. The method of claim 27, wherein X is C(O)—NH and each of the A¹ to Aⁱ is an amino acid residue selected from the group consisting of natural amino acids, unnatural amino acids, and modified amino acids.

30. The method of claim 27, wherein the compound has the formula:

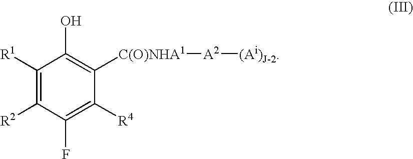

31. The method of claim 27, wherein the compound has the formula:
R—C(O)—NH-A (IV),
wherein:
A is an amino acid residue selected from the group consisting of natural amino acids, unnatural amino acids, and modified amino acids.

32. The method of claim 27, wherein the compound has the formula: 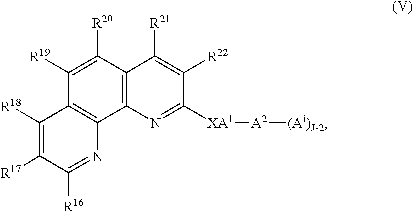
wherein:
R¹⁶ to R²² is independently selected from the group consisting of are independently selected from the group consisting of H, halogen, —NO₂, —CN, —C(O)ₘR⁵, —C(O)NR⁶R⁷, —S(O)ₜR⁸, —SO₂NR⁹R¹⁰, —OR¹¹, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS; and
R⁵, R⁶, R⁷, R⁸, R⁹, R¹⁰, and R¹¹ are independently selected from the group consisting of H, halogen, substituted or unsubstituted alkyl, and substituted or unsubstituted heteroalkyl.

33. The method of claim 32, wherein the compound has the formula:

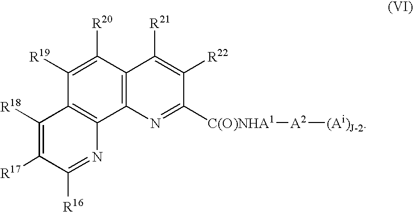

34. The method of claim 27, wherein the members of the library are attached to a solid support.

35. The method of claim 27, wherein the fluorogenic moiety is a 5-fluorosalicylic acid moiety.

36. The method of claim 27, wherein the fluorogenic moiety is a phenanthroline carboxylic acid moiety.

37. The method of claim 27, wherein the lanthanide is terbium, europium, samarium, dysprosium, or gadolinium.

38. The method of claim 27, wherein the enzyme is a protease or an esterase.

39. A method for detecting the presence of an enzyme in a sample, wherein the enzyme has a known peptide sequence as the enzyme substrate, comprising the steps of:
(a) contacting the sample with a compound of claim 1 at the presence of a lanthanide ion under conditions permissible for the enzyme activity, wherein the compound comprises the known peptide sequence as the enzyme substrate that; and
(b) detecting change in fluorescence or magnetic resonance contrast, wherein the an increase in fluorescence or magnetic resonance contrast indicates the presence of the enzyme in the sample.

40. The method of claim 39, wherein the compound has the formula: 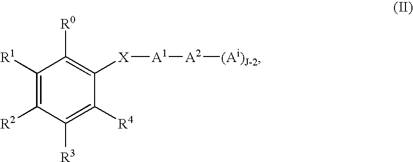
wherein:
R¹, R², R³, and R⁴ are independently selected from the group consisting of H, halogen, —NO₂, —CN, —C(O)ₘR⁵, —C(O)NR⁶R⁷, —S(O)ₜR⁸, —SO₂NR⁹R¹⁰, —OR¹¹, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS;
R⁰, R⁵, R⁶, R⁷, R⁸, R⁹, R¹⁰, and R¹¹ are independently selected from the group consisting of H, halogen, substituted or unsubstituted alkyl, and substituted or unsubstituted heteroalkyl;
R¹² is either present or absent, and when present, is a member selected from the group consisting of substituted or unsubstituted alkyl, and substituted or unsubstituted heteroalkyl; when R¹² is absent, Y is attached directly to the detectable moiety;
R¹³ is a linking group adjoining the detectable moiety and the solid support;
m is a member selected from the group consisting of the integers from 1 to 2;
t is a member selected from the group consisting of the integers from 0 to 2;
Y is an organic functional group or methyl, and is preferably a member selected from the group consisting of —COOR¹⁴, CONR¹⁴R¹⁵, —C(O)R¹⁴, —OR¹⁴, —SR¹⁴, NR¹⁴R¹⁵, —C(O)NR¹⁴R¹⁵, and —C(O)SR¹⁴;
R¹⁴ and R¹⁵ are members independently selected from the group consisting of H, substituted or unsubstituted alkyl, substituted or unsubstituted heteroalkyl, substituted or unsubstituted aryl and substituted or unsubstituted heteroaryl; and
SS is a solid support.

41. The method of claim 39, wherein X is C(O)—NH and each of the A¹ to Aⁱ is an amino acid residue selected from the group consisting of natural amino acids, unnatural amino acids, and modified amino acids.

42. The method of claim 39, wherein the compound has the formula:

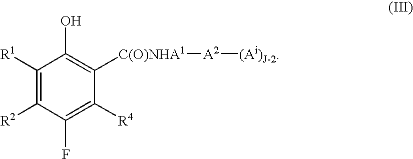

43. The method of claim 39, wherein the compound has the formula:
R—C(O)—NH-A (IV),
wherein:
A is an amino acid residue selected from the group consisting of natural amino acids, unnatural amino acids, and modified amino acids.

44. The method of claim 39, wherein the compound has the formula: 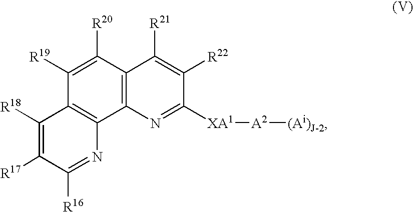
wherein:
R¹⁶ to R²² is independently selected from the group consisting of are independently selected from the group consisting of H, halogen, —NO₂, —CN, —C(O)ₘR⁵, —C(O)NR⁶R⁷, —S(O)ₜR⁸, —SO₂NR⁹R¹⁰, —OR¹¹, substituted or unsubstituted alkyl, substituted or unsubstituted heteroalkyl, substituted or unsubstituted aryl, substituted or unsubstituted heteroaryl, substituted or unsubstituted heteroalkyl, —NH—C(O)—P, R¹²—Y, and —R¹³—SS; and
R⁵, R⁶, R⁷, R⁸, R⁹, R¹⁰, and R¹¹ are independently selected from the group consisting of H, halogen, substituted or unsubstituted alkyl, and substituted or unsubstituted heteroalkyl.

45. The method of claim 44, wherein the compound has the formula:

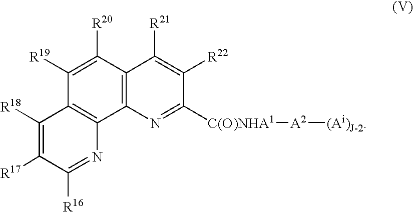

46. The method of claim 39, wherein the compound is attached to a solid support.

47. The method of claim 39, wherein the fluorogenic moiety is a 5-fluorosalicylic acid moiety.

48. The method of claim 39, wherein the fluorogenic moiety is a phenanthroline carboxylic acid moiety.

49. The method of claim 39, wherein the lanthanide is terbium, europium, samarium, dysprosium, or gadolinium.

50. The method of claim 39, wherein the enzyme is a protease or an esterase.

51. The method of claim 39, further comprising a step of quantifying the increase in fluorescence or magnetic resonance contrast, thereby determining the amount of the enzyme present in the sample.
